(19) United States
(12) Patent Application Publication
(10) Pub. No.: US 2024/0344007 A1
(43) Pub. Date: Oct. 17, 2024

(54) DEVICES FOR GROWING CULTURES OF MICROORGANISMS

(71) Applicant: The Regents of the University of California, Oakland, CA (US)

(72) Inventor: Alexander Groisman, San Diego, CA (US)

(21) Appl. No.: 18/294,270

(22) PCT Filed: Aug. 5, 2022
(86) PCT No.: PCT/US2022/074605

§ 371 Date: Feb. 1, 2024

Related U.S. Application Data
(60) Provisional application No. 63/230,618, filed on Aug. 6, 2021.

(51) Int. Cl. C12M 1/12; C12M 1/32

(52) U.S. Cl. CPC C12M 25/02; C12M 23/12

(57) ABSTRACT

Compositions and methods are described herein that are useful for microbial culture of “uncultivable” microorganisms. The disclosure specifically ad-dresses the issues pertaining to efficient extraction of the cultivated microorganisms from growth chambers. The compositions and methods described herein are particularly useful for liquid medium but are also useful for solid media such as hydrogels or any such comparable media. In addition, the disclosure relates to, among other things, preventing cross-contamination and contamination from environmental microorganisms, especially during incubation in various natural and man-made environments and during extraction from growth chambers. Since these uncultivatable microorganisms are not initially cultured in synthetic laboratory medium, more care may be necessary to maintain sterility, and prevent the cross-contamination with environmental microorganisms.

### CROSS-REFERENCE TO RELATED APPLICATIONS

This application claims the priority of U.S. provisional application Ser. No. 63/230,618 filed Aug. 6, 2021, the disclosure of which is incorporated herein by reference in its entirety.

### BACKGROUND

Microbial organisms produce a wide variety of unique secondary metabolites, small organic molecules, including molecules that can be practically useful, e.g., as antibiotics. Only a small fraction of microorganisms found on the planet can be readily cultured in a synthetic medium in the laboratory, however. The rest are called “uncultivatable” microorganisms. The isolation, purification, propagation, and culturing of such uncultivatable microorganisms in the laboratory can facilitate the exploration of the secondary metabolites they produce and open the way to the discovery of new antibiotics and other drugs.

### SUMMARY

Compositions and methods are described herein that are useful for microbial culture of “uncultivable” microorganisms. The disclosure specifically addresses the issues pertaining to efficient extraction of the cultivated microorganisms from growth chambers. The compositions and methods described herein are particularly useful for liquid medium but are also useful for solid media such as hydrogels or any such comparable media. In addition, the disclosure relates to, among other things, preventing cross-contamination and contamination from environmental microorganisms, especially during incubation in various natural and man-made environments and during extraction from growth chambers. Since these uncultivatable microorganisms are not initially cultured in synthetic laboratory medium, more care may be necessary to maintain sterility, and prevent the cross-contamination with environmental microorganisms.

### DESCRIPTION OF THE FIGURES UPDATE

Aspects of the present disclosure can be better understood with reference to the following drawings. The components in the drawings are not necessarily to scale. Instead, emphasis is placed on clearly illustrating the principles and methods underlying the present disclosure. Furthermore, components can be shown as transparent in certain views for clarity of illustration only and not to indicate that the illustrated component is necessarily transparent.

FIGS. 1-4, 5A, 5B, 6-10, 13-15, 16A, 16B, 17, 18A, 18B, and 19 are schematics of device examples according to the disclosure having a central plate with an array of holes or wells and, among other features, one or more porous membranes, one or more perforated films, one or more thin films that can be removable, and the like.

FIG. 11 is a schematic of exemplary adhesion patterns between, for example, the perforated film and central plate and between the central plate and porous membrane. These are examples of adhesion patterns that can reduce, for example, the area of adhesion between the central plate and porous membrane and the central plate and perforated film.

FIGS. 12A and 12B are schematics of microfabricated chips that can be used in the devices according to the disclosure.

Like reference numbers in the various figures indicate like elements. Some elements may be present in identical or equivalent multiples: in such cases only one or more representative elements may be designated by a reference number but it will be understood that such reference numbers apply to all such identical elements. Unless otherwise indicated, all figures and drawings in this document are not to scale and are chosen for the purpose of illustrating different embodiments of the invention. In particular the dimensions of the various components are depicted in illustrative terms only, and no relationship between the dimensions of the various components should be inferred from the drawings, unless so indicated. Although terms such as “top”, “bottom”, “upper”, “lower”, “under”, “over”, “front”, “back”, “up” and “down”, and “first” and “second” may be used in this disclosure, it should be understood that those terms are used in their relative sense only unless otherwise noted.

### DETAILED DESCRIPTION

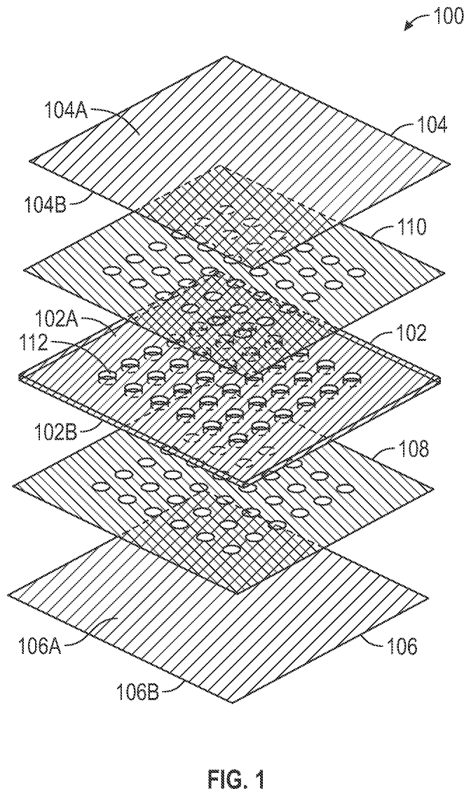

Making reference to FIG. 1, the disclosure relates to a device 100 comprising a central plate 102 having a central plate first major surface 102A and a central plate second major surface 102B, the central plate 102 comprising a plurality of holes 112 extending from the central plate first major surface 102A to the central plate second major surface 102B;
a first porous membrane 104 having a first porous membrane first surface 104A, and a first porous membrane second surface 104B, the first porous membrane second surface 104B adhered to the central plate first major surface 102A and sealing at least a portion of the plurality of holes 112 on the central plate first major surface 102A, wherein the first porous membrane is impassable for microorganisms; and
a second porous membrane 106 having a second porous membrane first surface 106A, and a second porous membrane second surface 106B, the second porous membrane first surface 106A adhered to the central plate second major surface 102B and sealing at least a portion of the plurality of holes 112 on the central plate second major surface 102B, wherein the second porous membrane is impassable for microorganisms. FIG. 1, and the figures that follow, also show adhesive layers 108 and 110, which can be pressure-sensitive adhesives, for example, pressure sensitive adhesive tapes (e.g., 467MP or 468Mp by 3M). But other forms of adhesion known in the art are also contemplated herein.

A hole or well on a surface of the central plate is considered to be “sealed” by a porous membrane adhered to the surface of the plate, when this hole or the well is completely enclosed by a region, where the space between the membrane and the surface of the central plate is inaccessible to microorganisms from the hole or well, from any other holes or well on the central plate, and from the outside environment. On the other hand, the sealing of the hole or well by a porous membrane does not block the flow or diffusion through pores in the porous membrane between the hole or well in the plate and the outside environment.

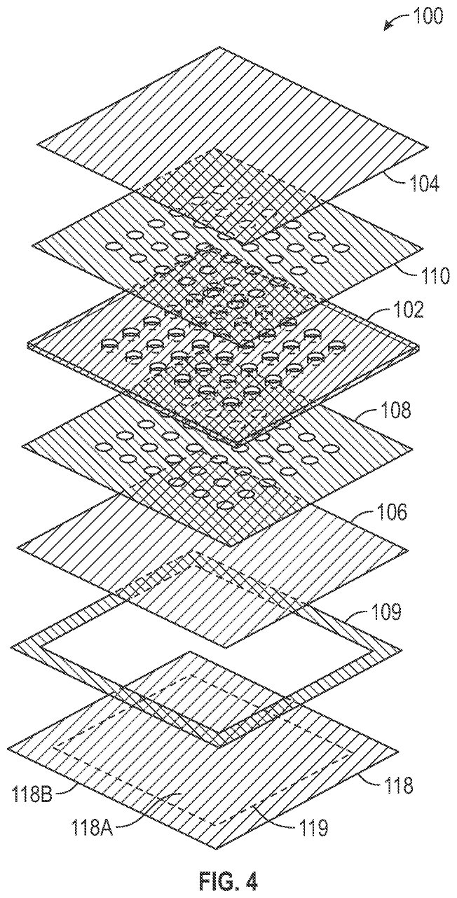

The construction described in FIG. 1 is comprised in other devices described herein, including the devices described in FIGS. 4, 7, and 10 herein. The device 100 described in FIG. 4 further comprises a third porous membrane 118 having a third porous membrane first surface 118A, and a third porous membrane second surface 118B, the third porous membrane first surface 118A adhered to the second porous membrane second surface 106B and covering an area with at least a portion of the plurality of holes 112 on the central plate second major surface 102B that are sealed by the second porous membrane 106, wherein the third porous membrane 118 is impassable for microorganisms; and at least one sealed and sterile volume between the second porous membrane second surface 106B and third porous membrane first surface 118A, wherein at least a portion of the at least one sealed and sterile volume is adjacent to at least one hole 112 on the central plate second major surface 102B that is sealed by the second porous membrane 106. FIG. 4 (and others herein) has a “cutting line” 119 that is simply meant to identify a line on, for example, a porous membrane, along which one can cut the porous membrane to remove it from the device for the reasons described herein (for example, to access the sterile volume between the second porous membrane second surface 106B and the third porous membrane first surface 118A).

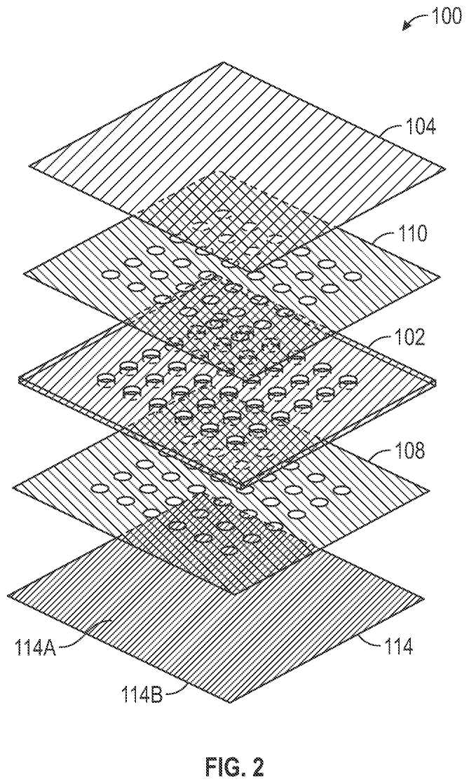

Making reference to FIG. 2, the disclosure relates to a device 100 comprising:
a central plate 102 having a central plate first major surface 102A and a central plate second major surface 102B, the central plate 102 comprising a plurality of holes 112 extending from the central plate first major surface 102A to the central plate second major surface 102B; and
a first porous membrane 104 having a first porous membrane first surface 104A, and a first porous membrane second surface 104B, the first porous membrane second surface 104B adhered to the central plate first major surface 102A and sealing at least a portion of the plurality of holes 112 on the central plate first major surface 102A, wherein the first porous membrane is impassable for microorganisms; and
a thin film 114, which may be pierceable, having a thin film first surface 114A and a thin film second surface 114B, the thin film first surface 114A adhered to the central plate second major surface 102B and sealing at least a portion of the plurality of holes 112 on the central plate second major surface 102B;
wherein:
the thin film second surface 114B can be cleaned and sterilized, and the thin film 114 can be pierced in an area adjacent to one hole 112 or a plurality of holes 112 on the central plate without breaking the sealing of other holes on the central plate second major surface 102B.

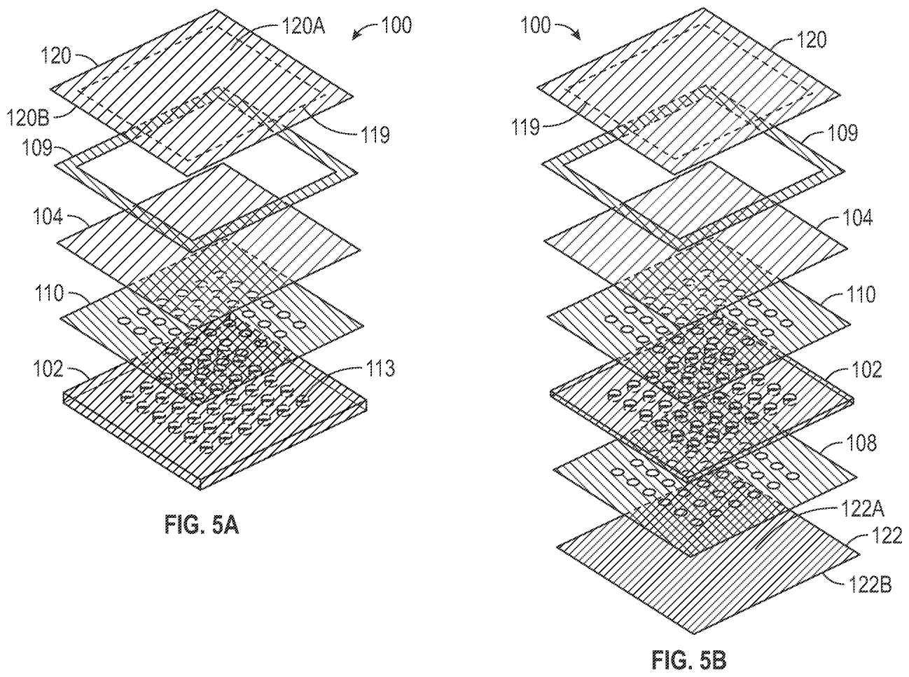

Making reference to FIG. 5A, the disclosure also relates to a device 100 comprising:
a central plate 102 having a central plate first major surface 102A and a central plate second major surface 102B, the central plate 102 comprising a plurality of wells 113 on the second plate first major surface 102A;
a first porous membrane 104 having a first porous membrane first surface 104A, and a first porous membrane second surface 104B, the first porous membrane second surface 104B adhered to the central plate first major surface 102A and sealing at least a portion of the plurality of wells 113 on the central plate first major surface 102A, wherein the first porous membrane 104 is impassable for microorganisms;
a fourth porous membrane 120 having a fourth porous membrane first surface 120A, and a fourth porous membrane second surface 120B, the fourth porous membrane second surface 120B adhered to the first porous membrane first surface 104A and covering an area with at least a portion of the plurality of wells 113 on the central plate first major surface 102A that are sealed by the first porous membrane 104, wherein the fourth porous membrane 120 is impassable for microorganisms; and
at least one sealed and sterile volume between the first porous membrane first surface 104A and second porous membrane second surface 120B, wherein at least a portion of the at least one sealed and sterile volume is adjacent to at least one well 113 on the central plate first major surface 102A that is sealed by the first porous membrane 104,

The wells 113 on the central plate 102 in the figures described herein, including FIG. 5A, are similar to the holes 112 except that they are sealed at the central plate second major surface 102B. In this example, the central plate is a solid central plate second major surface 102B that seals the central plate 102 such that the central plate 102 has wells and not holes a plurality of holes 112 extending from the central plate first major surface 102A to the central plate second major surface 102B.

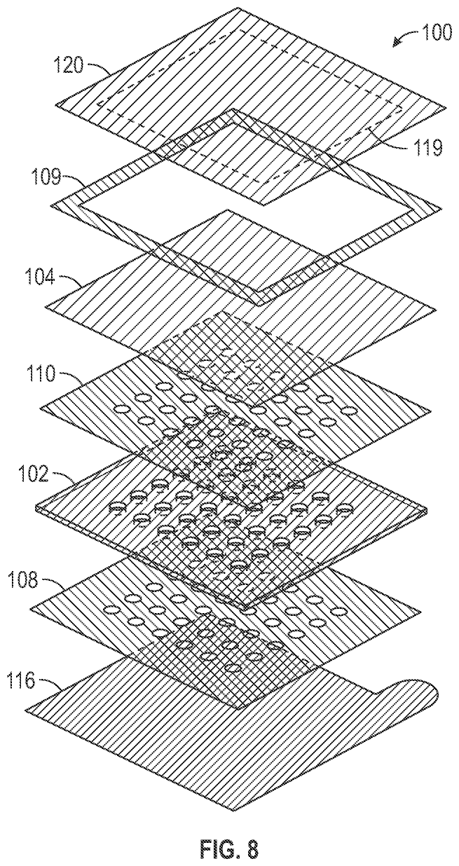

Making reference to FIG. 8, the disclosure also relates to a device 100 comprising:
a central plate 102 having a central plate first major surface 102A and a central plate second major surface 102B, the central plate 102 comprising a plurality of holes 112;
a first porous membrane 104 having a first porous membrane first surface 104A, and a first porous membrane second surface 104B, the first porous membrane second surface 104B adhered to the central plate first major surface 102A and sealing at least a portion of the plurality of holes 112 on the central plate first major surface 102A, wherein the first porous membrane 104 is impassable for microorganisms;
a fourth porous membrane 120 having a fourth porous membrane first surface 120A, and a fourth porous membrane second surface 120B, the fourth porous membrane second surface 120B adhered to the first porous membrane first surface 104A and covering an area with at least a portion of the plurality of wells 113 on the central plate first major surface 102A that are sealed by the first porous membrane 104, wherein the fourth porous membrane 120 is impassable for microorganisms;
at least one sealed and sterile volume between the first porous membrane first surface 104A and second porous membrane second surface 120B, wherein at least a portion of the at least one sealed and sterile volume is at least one hole 112 on the central plate first major surface 102A that is sealed by the first porous membrane 104; and
a removable film 116 (interchangeably referred to herein as a “tear-off film”) having a removable film first surface 116A and a removable film second surface 116B, the removable film first surface 116A adhered to the central plate second major surface 102B.

Making reference to FIG. 5B, the disclosure also relates to a device 100 comprising:
a central plate 102 having a central plate first major surface 102A and a central plate second major surface 102B, the central plate 102 comprising a plurality of holes 112 extending from the central plate first major surface 102A to the central plate second major surface 102B;
a first porous membrane 104 having a first porous membrane first surface 104A, and a first porous membrane second surface 104B, the first porous membrane second surface 104B adhered to the central plate first major surface 102A and sealing at least a portion of the plurality of holes 112 on the central plate first major surface 102A, wherein the first porous membrane 104 is impassable for microorganisms;
a fourth porous membrane 120 having a fourth porous membrane first surface 120A, and a fourth porous membrane second surface 120B, the fourth porous membrane second surface 120B adhered to the first porous membrane first surface 104A and covering an area with at least a portion of the plurality of holes 112 on the central plate first major surface 102A that are sealed by the first porous membrane 104, wherein the fourth porous membrane 120 is impassable for microorganisms;
at least one sealed and sterile volume between the first porous membrane first surface 104A and fourth porous membrane second surface 120B, wherein at least a portion of the at least one sealed and sterile volume is adjacent to at least one hole 112 on the central plate first major surface 102A that is sealed by the first porous membrane 104; and
a solid sheet 122 having a solid sheet first surface 122A and a solid sheet second surface 122B, the solid sheet first surface 122A adhered to the central plate second major surface 102B and sealing at least a portion of the plurality of holes 112 on the central plate second major surface 102B.

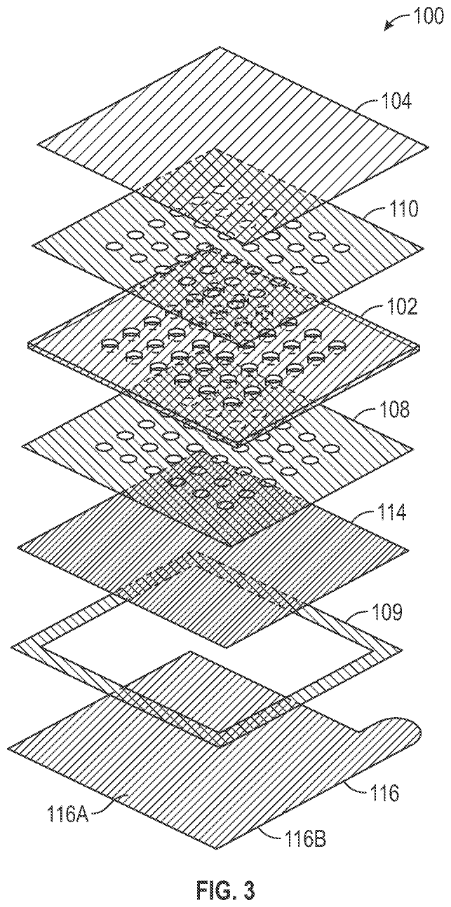

Making reference to FIG. 3, the disclosure also relates to a device 100 comprising:
a central plate 102 having a central plate first major surface 102A and a central plate second major surface 102B, the central plate 102 comprising a plurality of holes 112 extending from the central plate first major surface 102A to the central plate second major surface 102B;
a first porous membrane 104 having a first porous membrane first surface 104A, and a first porous membrane second surface 104B, the first porous membrane second surface 104B adhered to the central plate first major surface 102A and sealing at least a portion of the plurality of holes 112 on the central plate first major surface 102A, wherein the first porous membrane is impassable for microorganisms;
a thin film 114 having a thin film first surface 114A and a thin film second surface 114B, the thin film first surface 114A adhered to the central plate second major surface 102B and sealing at least a portion of the plurality of holes 112 on the central plate second major surface 102B, wherein the thin film 114 can be pierced in an area adjacent to one hole 112 or a plurality of holes 112 on the central plate 102 without breaking the sealing of other holes 112 on the central plate second major surface 102B by the thin film 114;
a removable film 116 having a removable film first surface 116A and a removable film second surface 116B, the removable film first surface 116A adhered to via adhesive frame 109 (e.g., a frame made of a pressure sensitive adhesive transfer tape, e.g., 467MP by 3M) to the thin film second surface 114B and covering an area with at least a portion of the plurality of holes 112 on the central plate second major surface 102B that are sealed by the thin film 114; at least one sealed and sterile volume between the thin film second surface 114B and the removable film first surface 116A, and at least a portion of the at least one sealed and sterile volume is adjacent to at least one hole 112 on the central plate second major surface 102B that is sealed by the thin film 114; and
the removable film 116 can be separated from the thin film 114 without unsealing the sealing of the holes on the central plate second major surface 102B that are sealed by the thin film 114.

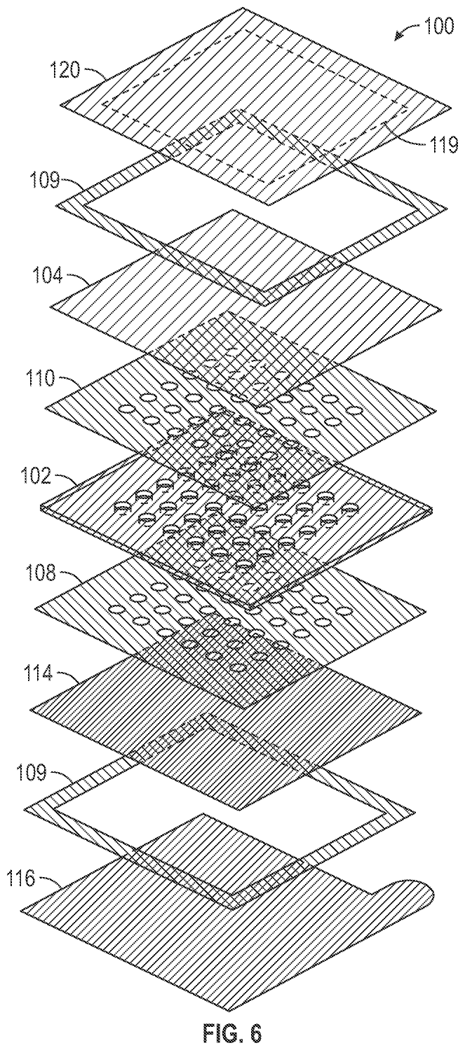

The device 100 described in FIG. 6 is similar to the device described in FIG. 3 except that the device described in FIG. 6 further comprises a fourth porous membrane 120 having a fourth porous membrane first surface 120A, and a fourth porous membrane second surface 120B, the fourth porous membrane second surface 120B adhered to the first porous membrane first surface 104A and covering an area with at least a portion of the plurality of holes 112 on the central plate first major surface 102A that are sealed by the first porous membrane 104, wherein the fourth porous membrane 120 is impassable for microorganisms.

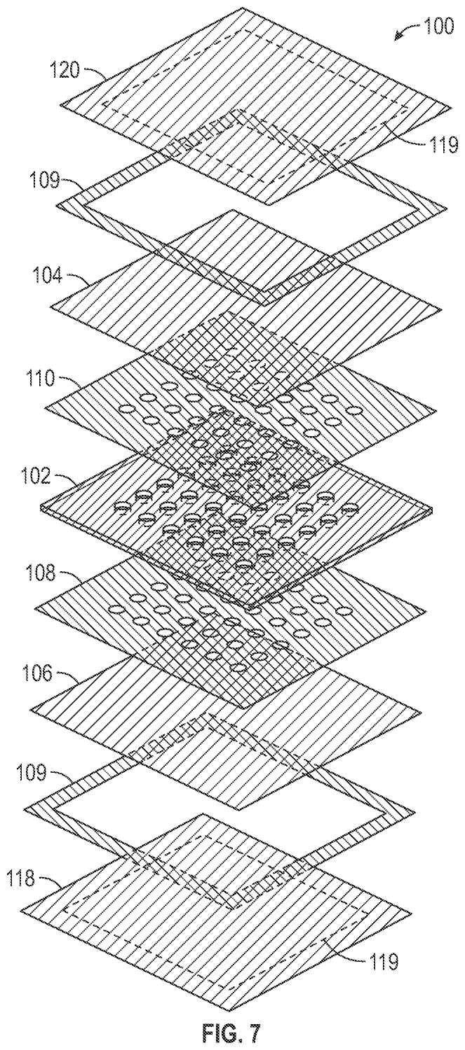

The device 100 described in FIG. 7 is similar to the device described in FIG. 6 except that the thin film 114 and the removable film 116 are replaced by a second porous membrane 106 and a third porous membrane 118, respectively, wherein at least one sealed and sterile volume exists between the second porous membrane second surface 106B and third porous membrane first surface 118A and at least a portion of the at least one sealed and sterile volume is adjacent to at least one hole 112 on the central plate second major surface 102B that is sealed by the second porous membrane 106.

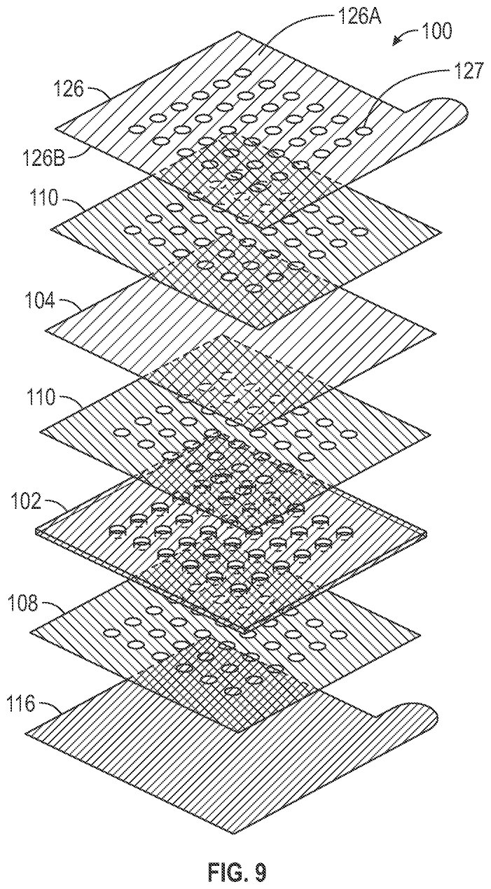

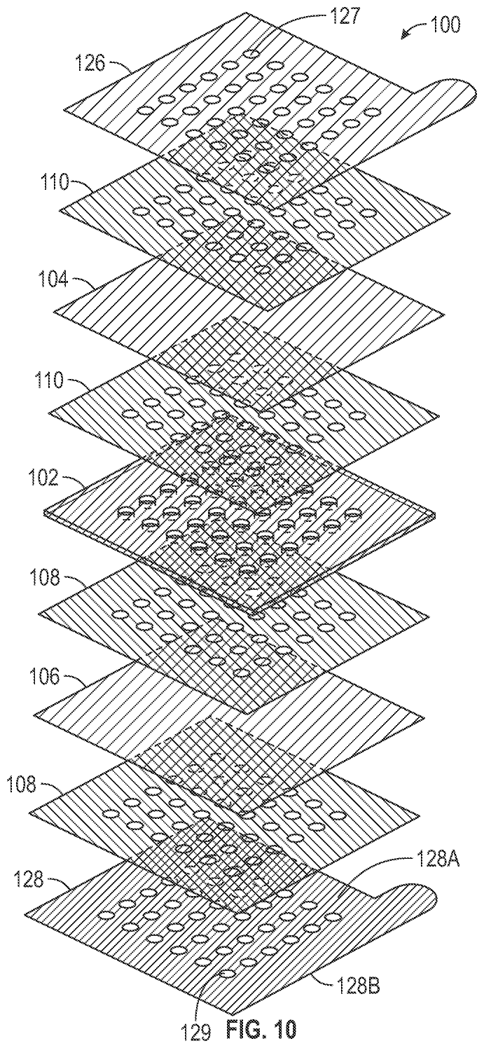

Making reference to FIGS. 9 and 10, the devices 100 depicted there comprise:
a central plate 102 having a central plate first major surface 102A and a central plate second major surface 102B, the central plate 102 comprising a plurality of holes 112 extending from the central plate first major surface 102A to the central plate second major surface 102B;
a first porous membrane 104 having a first porous membrane first surface 104A, and a first porous membrane second surface 104B, the first porous membrane second surface 104B adhered to the central plate first major surface 102A and sealing at least a portion of the plurality of holes 112 on the central plate first major surface 102A, wherein the first porous membrane is impassable for microorganisms; and
a perforated film 126 having a perforated film first surface 126A and a perforated film second surface 126B, the perforated film second surface 126B adhered to the first porous membrane first surface 104A, the perforated film 126 having a plurality of holes 129 and at least a portion of the holes 129 in the perforated film 126 at least partially overlap with at least a portion of the holes 102 on the central plate first major surface 102A;
wherein the first porous membrane 104 together with the perforated film 126 adhered to it can be separated from the central plate 102.

The device 100 in FIG. 9 further comprises a removable film 116 having a removable film first surface 116A and a removable film second surface 116B, the removable film first surface 116A adhered to the central plate second major surface 102B and sealing at least a portion of the plurality of holes 112 on the central plate second major surface 102B.

The device 100 in FIG. 10 further comprises a second porous membrane 106 having a second porous membrane first surface 106A, and a second porous membrane second surface 106B, the second porous membrane first surface 106A adhered to the central plate second major surface 102B and sealing at least a portion of the plurality of holes 112 on the central plate second major surface 102B, wherein the second porous membrane is impassable for microorganisms; and
a second perforated film 128 having a second perforated film first surface 128A and a second perforated film second surface 128B, the second perforated film first surface 128A adhered to the second porous membrane second surface 106B, the second perforated film 128 having a plurality of holes 129 and at least a portion of the holes 129 in the second perforated film 128 at least partially overlap with at least a portion of the holes 102 on the central plate second major surface 102B. In this example, the second porous membrane 106 together with the second perforated film 128 adhered to it can be separated from the central plate 102.

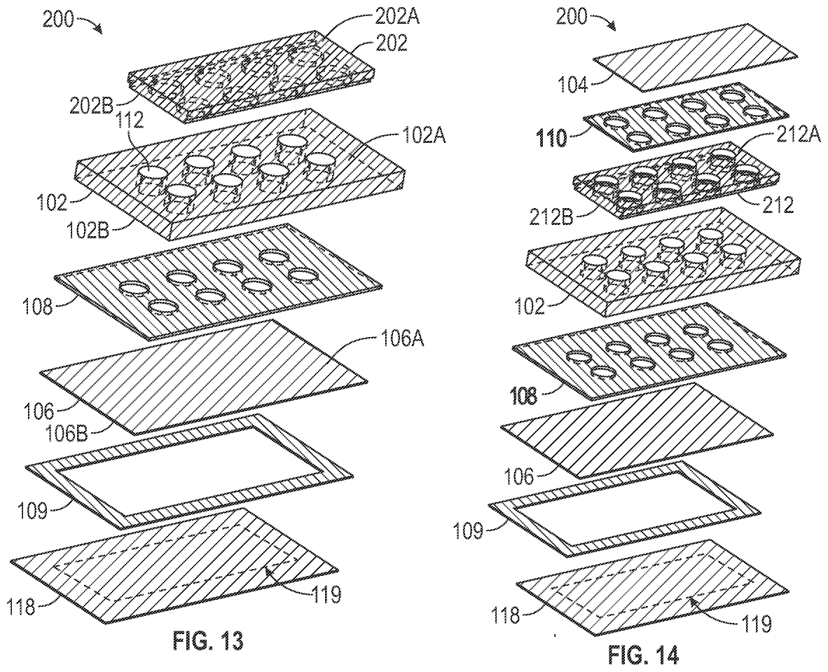

The devices described in FIGS. 13-19 are devices that are characterized herein as “self-inoculating.” As used herein, the term “self-inoculating” generally refers to devices where the inoculation of the culture chambers does not occur as a result of a process when the central plate holes 112 or wells 113 are deliberately filled with a suspension of microorganisms. Instead, the self-inoculating devices have culture chambers that can be accessed by microorganisms from the outside (for example, via the capillaries 206 or 216). The holes 112 or wells 113 in the central plates of these devices are initially filled with a sterile medium, and the devices are placed into an environment containing microorganisms. The culture chambers in these devices are populated with microorganisms as a result of the environmental microorganisms spontaneously penetrating into the culture chambers and proliferating in the culture chambers (the holes 112 and wells 113 in the central plate).

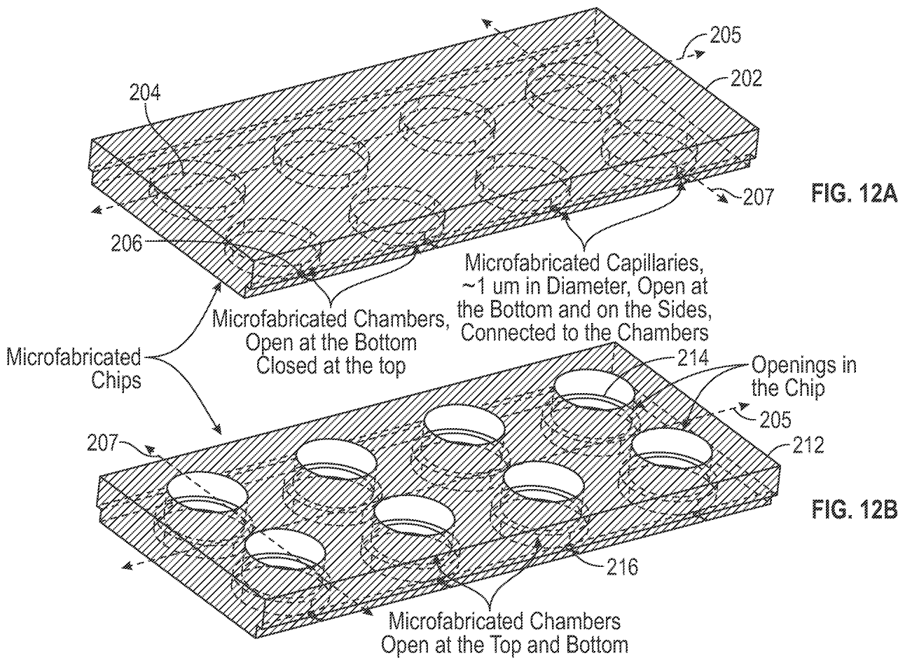

Making reference to FIG. 13, the disclosure relates to a device 200 comprising a central plate 102 having a central plate first major surface 102A and a central plate second major surface 102B, the central plate 102 comprising a plurality of holes 112 extending from the central plate first major surface 102A to the central plate second major surface 102B;
a second porous membrane 106 having a second porous membrane first surface 106A, and a second porous membrane second surface 106B, the second porous membrane first surface 106A adhered to the central plate second major surface 102B and sealing at least a portion of the plurality of holes 112 on the central plate second major surface 102B, wherein the second porous membrane is impassable for microorganisms
a third porous membrane 118 having a third porous membrane first surface 118A, and a third porous membrane second surface 118B, the third porous membrane first surface 118A adhered to the second porous membrane second surface 106B and covering an area with at least a portion of the plurality of holes 112 on the central plate second major surface 102B that are sealed by the second porous membrane 106, wherein the third porous membrane 118 is impassable for microorganisms;
at least one sealed and sterile volume between the second porous membrane second surface 106B and third porous membrane first surface 118A, wherein at least a portion of the at least one sealed and sterile volume is adjacent to at least one hole 112 on the central plate second major surface 102B that is sealed by the second porous membrane 106; and
a microfabricated chip 202 (see FIG. 12A) having a microfabricated chip first major surface 202A, a microfabricated chip second major surface 202B, a first major axis 205 and a second major axis 207 perpendicular to the first major axis 205, a plurality of chambers 204 having an opening on the microfabricated chip second major surface 202B and extending from the microfabricated chip second major surface 202B in the direction of, but not through to the microfabricated chip first major surface 202A, each of the plurality of chambers 204 comprising at least one microfabricated capillary 206 (for example, measuring from about 0.2-2 μm in diameter: not shown) radiating from the chamber substantially in the direction substantially parallel to the second major axis 207, wherein each of the at least one microfabricated capillary 206 fluidically connects each of the plurality of chambers 204 with an environment outside of each of the plurality of chambers 204 and wherein the microfabricated chip second major surface 202B is adhered to the central plate first major surface 102A;
wherein the opening of each of the plurality of chambers 204 at least partially overlaps with at least a portion of at least one hole 112 on the central plate first major surface 102A.

The device 200 described in FIG. 14 is similar to the device described in FIG. 13 except that the device described in FIG. 14 comprises a microfabricated chip 212 (see FIG. 12B) instead of microfabricated chip 202, microfabricated chip 212 having a microfabricated chip first major surface 212A, a microfabricated chip second major surface 212B, a first major axis 205 and a second major axis 207 perpendicular to the first major axis 205, a plurality of chambers 214 extending from the microfabricated chip second major surface 212B through to the microfabricated chip first major surface 212A, each of the plurality of chambers 214 comprising at least one microfabricated capillary 216 (not shown) radiating from the chamber substantially in the direction of the second major axis 207, wherein each of the at least one microfabricated capillary 216 fluidically connects each of the plurality of chambers 214 with an environment outside of each of the plurality of chambers 214, wherein each of the plurality of chambers 214 at least partially overlaps with at least one hole 112 on the central plate first major surface 102A and wherein the microfabricated chip second major surface 212B is adhered to the central plate first major surface 102A; and
a first porous membrane 104 having a first porous membrane first surface 104A, and a first porous membrane second surface 104B, the first porous membrane second surface 104B adhered to the microfabricated chip first major surface 212A, wherein the first porous membrane is impassable for microorganisms.

In any device 200 described herein, comprising a microfabricated chip 202 or 212, not all of the chambers 204 or 214 need to have microfabricated capillaries 206 or 216. In other words, some, but not all of the chambers 204 or 214 can comprise capillaries 206 or 216, respectively. In some instances, however, all of the all of the chambers 204 or 214 can comprise capillaries 206 or 216, respectively.

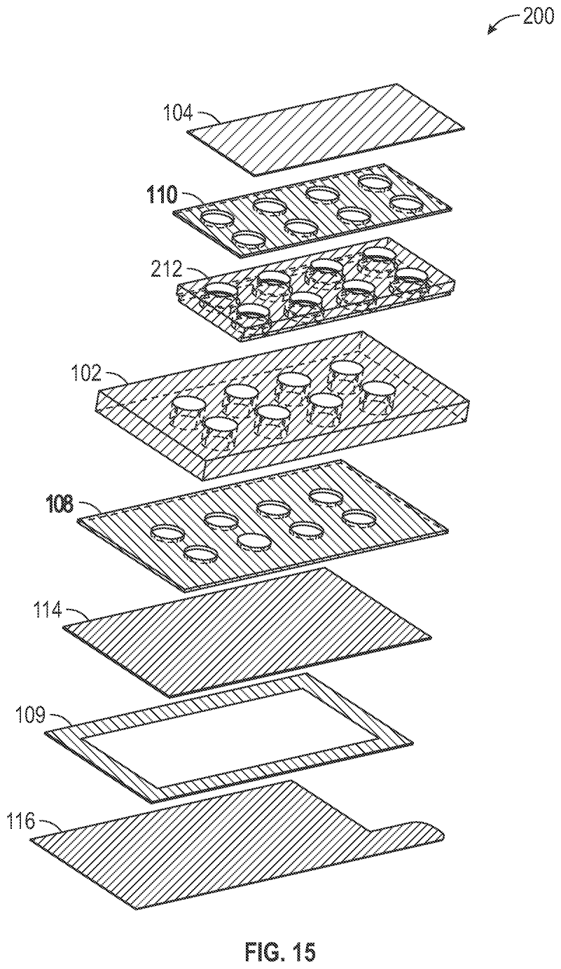

Making reference to FIG. 15, the disclosure relates to a device 200 comprising a central plate 102 having a central plate first major surface 102A and a central plate second major surface 102B, the central plate 102 comprising a plurality of holes 112 extending from the central plate first major surface 102A to the central plate second major surface 102B;
a microfabricated chip 212 having a microfabricated chip first major surface 212A, a microfabricated chip second major surface 212B, a first major axis 205 and a second major axis 207 perpendicular to the first major axis 205, a plurality of chambers 214 extending from the microfabricated chip second major surface 212B through to the microfabricated chip first major surface 212A, each of the plurality of chambers 214 comprising at least one microfabricated capillary 216 (not shown) radiating from the chamber substantially in the direction of the second major axis 207, wherein each of the at least one microfabricated capillary 216 fluidically connects each of the plurality of chambers 214 with an environment outside of each of the plurality of chambers 214, wherein each of the plurality of chambers 214 at least partially overlaps with at least one hole 112 on the central plate first major surface 102A and wherein the microfabricated chip second major surface 212B is adhered to the central plate first major surface 102A;
a first porous membrane 104 having a first porous membrane first surface 104A, and a first porous membrane second surface 104B, the first porous membrane second surface 104B adhered to the microfabricated chip first major surface 212A and sealing at least a portion of the plurality of chambers 214 on the microfabricated chip first major surface 212A, wherein the first porous membrane is impassable for microorganisms;
a thin film 114 having a thin film first surface 114A and a thin film second surface 114B, the thin film first surface 114A adhered to the central plate second major surface 102B and sealing at least a portion of the plurality of holes 112 on the central plate second major surface 102B, wherein the thin film 114 can be pierced in an area adjacent to one hole 112 or a plurality of holes 112 on the central plate 102 without breaking the sealing of other holes 112 on the second major surface of the central plate 102B by the thin film 114;
a removable film 116 having a removable film first surface 116A and a removable film second surface 116B, the removable film first surface 116A adhered to the thin film second surface 114B and covering an area with at least a portion of the plurality of holes 112 on the central plate second major surface 102B that are sealed by the thin film 114; at least one sealed and sterile volume between the thin film second surface 114B and the removable film first surface 116A, and at least a portion of the at least one sealed and sterile volume is adjacent to at least one hole 112 on the central plate second major surface 102B that is sealed by the thin film 114; and
the removable film 116 can be separated from the thin film 114 without unsealing the sealing of the holes 112 on the central plate second major surface 102B that are sealed by the thin film 114.

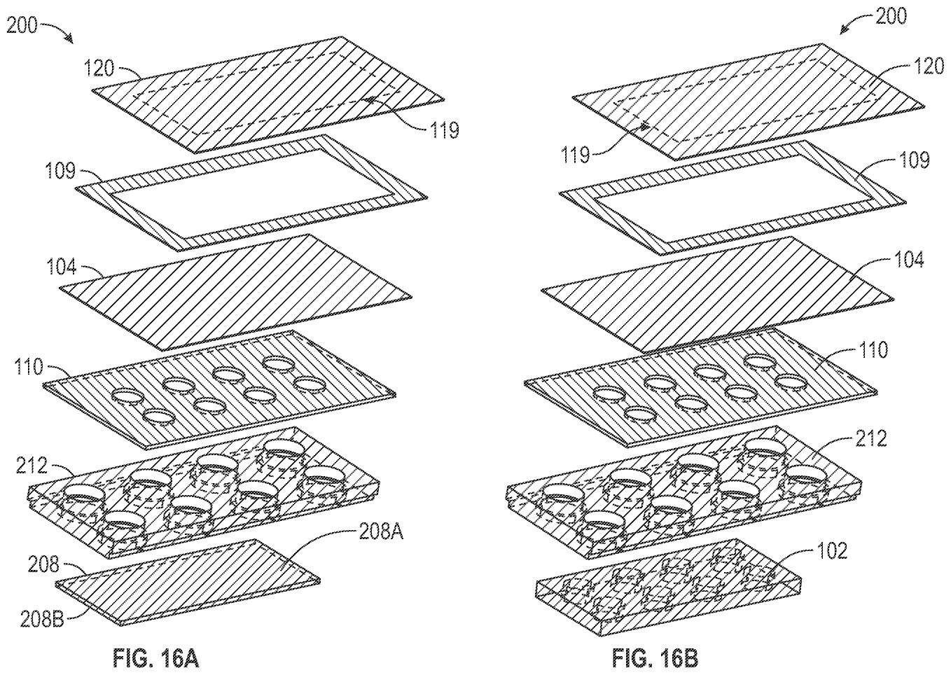

Making reference to FIG. 16A, the disclosure also relates to a device 200 comprising:
a solid substrate 208 having a solid substrate first major surface 208A and a solid substrate second major surface 208B;
a microfabricated chip 212 having a microfabricated chip first major surface 212A, a microfabricated chip second major surface 212B, a first major axis 205 and a second major axis 207 perpendicular to the first major axis 205, a plurality of chambers 214 extending from the microfabricated chip second major surface 212B through to the microfabricated chip first major surface 212A, each of the plurality of chambers 214 comprising at least one microfabricated capillary 216 (not shown) radiating from the chamber substantially in the direction of the second major axis 207, wherein each of the at least one microfabricated capillary 216 fluidically connects each of the plurality of chambers 214 with an environment outside of each of the plurality of chambers 214, wherein the microfabricated chip second major surface 212B is adhered to the solid substrate first major surface 208A;
a first porous membrane 104 having a first porous membrane first surface 104A, and a first porous membrane second surface 104B, the first porous membrane second surface 104B adhered to the microfabricated chip first major surface 212A and sealing at least a portion of the plurality of chambers 214 on the microfabricated chip first major surface 212A, wherein the first porous membrane 104 is impassable for microorganisms;
a fourth porous membrane 120 having a fourth porous membrane first surface 120A, and a fourth porous membrane second surface 120B, the fourth porous membrane second surface 120B adhered to the first porous membrane first surface 104A and covering an area with at least a portion of the plurality of chambers 214 on the microfabricated chip first major surface 212A that are sealed by the first porous membrane 104, wherein the fourth porous membrane 120 is impassable for microorganisms; and
at least one sealed and sterile volume between the first porous membrane first surface 104A and second porous membrane second surface 120B, wherein at least a portion of the at least one sealed and sterile volume is adjacent to at least one chamber 214 on the microfabricated chip first major surface 212A that is sealed by the first porous membrane 104.

Making reference to FIG. 16B the disclosure relates to a device 200 comprising a central plate 102 having a central plate first major surface 102A and a central plate second major surface 102B, the central plate 102 comprising a plurality of wells 113;
a microfabricated chip 212 having a microfabricated chip first major surface 212A, a microfabricated chip second major surface 212B, a first major axis 205 and a second major axis 207 perpendicular to the first major axis 205, a plurality of chambers 214 extending from the microfabricated chip second major surface 212B through to the microfabricated chip first major surface 212A, each of the plurality of chambers 214 comprising at least one microfabricated capillary 216 (not shown) radiating from the chamber substantially in the direction of the second major axis 207, wherein each of the at least one microfabricated capillary 216 fluidically connects each of the plurality of chambers 214 with an environment outside of each of the plurality of chambers 214, wherein each of the plurality of chambers 214 at least partially overlaps with at least one well 113 on the central plate first major surface 102A and wherein the microfabricated chip second major surface 212B is adhered to the central plate first major surface 102A;
a first porous membrane 104 having a first porous membrane first surface 104A, and a first porous membrane second surface 104B, the first porous membrane second surface 104B adhered to the microfabricated chip first major surface 212A and sealing at least a portion of the plurality of chambers 214 on the microfabricated chip first major surface 212A, wherein the first porous membrane 104 is impassable for microorganisms;
a fourth porous membrane 120 having a fourth porous membrane first surface 120A, and a fourth porous membrane second surface 120B, the fourth porous membrane second surface 120B adhered to the first porous membrane first surface 104A and covering an area with at least a portion of the plurality of chambers 214 on the central plate first major surface 212A that are sealed by the first porous membrane 104, wherein the fourth porous membrane 120 is impassable for microorganisms; and
at least one sealed and sterile volume between the first porous membrane first surface 104A and second porous membrane second surface 120B, wherein at least a portion of the at least one sealed and sterile volume is adjacent to at least one chamber 214 on the microfabricated chip first major surface 212A that is sealed by the first porous membrane 104.

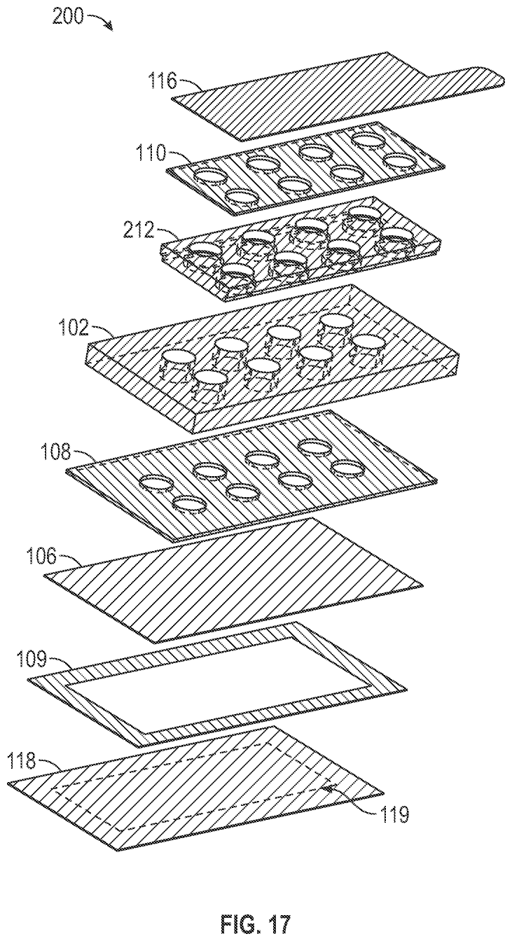

Making reference to FIG. 17, the disclosure relates to a device 200 comprising a central plate 102 having a central plate first major surface 102A and a central plate second major surface 102B, the central plate 102 comprising a plurality of holes 112 extending from the central plate first major surface 102A to the central plate second major surface 102B;
a microfabricated chip 212 having a microfabricated chip first major surface 212A, a microfabricated chip second major surface 212B, a first major axis 205 and a second major axis 207 perpendicular to the first major axis 205, a plurality of chambers 214 extending from the microfabricated chip second major surface 212B through to the microfabricated chip first major surface 212A, each of the plurality of chambers 214 comprising at least one microfabricated capillary 216 (not shown) radiating from the chamber substantially in the direction of the second major axis 207, wherein each of the at least one microfabricated capillary 216 fluidically connects each of the plurality of chambers 214 with an environment outside of each of the plurality of chambers 214, wherein each of the plurality of chambers 214 at least partially overlaps with at least one hole 112 on the central plate first major surface 102A and wherein the microfabricated chip second major surface 212B is adhered to the central plate first major surface 102A;
a removable film 116 having a removable film first surface 116A and a removable film second surface 116B, the removable film second surface 116A adhered to the microfabricated chip first major surface 212A;
a second porous membrane 106 having a second porous membrane first surface 106A, and a second porous membrane second surface 106B, the second porous membrane first surface 106A adhered to the central plate second major surface 102B and sealing at least a portion of the plurality of holes 112 on the central plate second major surface 102B, wherein the second porous membrane is impassable for microorganisms; and
a third porous membrane 118 having a third porous membrane first surface 118A, and a third porous membrane second surface 118B, the third porous membrane first surface 118A adhered to the second porous membrane second surface 106B and covering an area with at least a portion of the plurality of holes 112 on the central plate second major surface 102B that are sealed by the second porous membrane 106, wherein the third porous membrane 118 is impassable for microorganisms; and at least one sealed and sterile volume between the second porous membrane second surface 106B and third porous membrane first surface 118A, wherein at least a portion of the at least one sealed and sterile volume is adjacent to at least one hole 112 on the central plate second major surface 102B that is sealed by the second porous membrane 106.

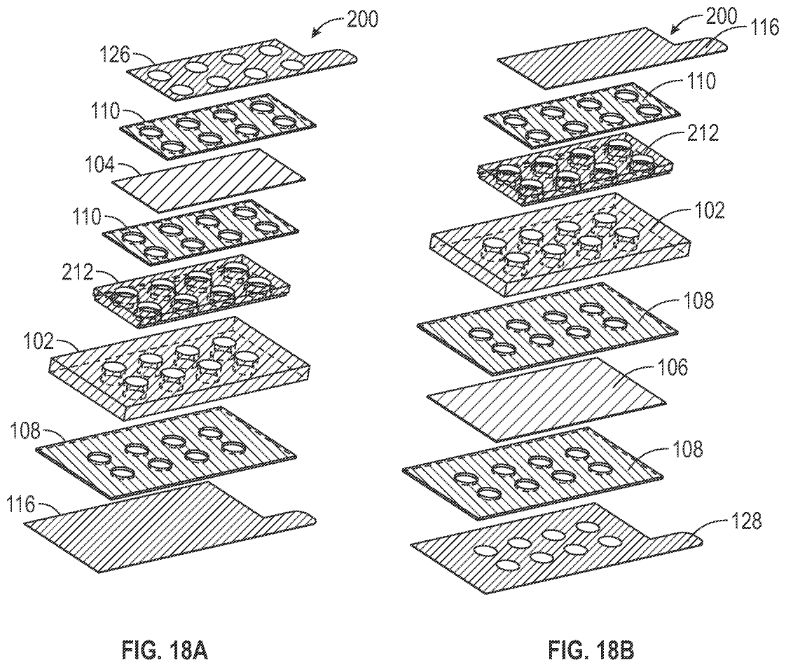

Making reference to FIG. 18A, the disclosure relates to a device 200 comprising a central plate 102 having a central plate first major surface 102A and a central plate second major surface 102B, the central plate 102 comprising a plurality of holes 112 extending from the central plate first major surface 102A to the central plate second major surface 102B;
a microfabricated chip 212 having a microfabricated chip first major surface 212A, a microfabricated chip second major surface 212B, a first major axis 205 and a second major axis 207 perpendicular to the first major axis 205, a plurality of chambers 214 extending from the microfabricated chip second major surface 212B through to the microfabricated chip first major surface 212A, each of the plurality of chambers 214 comprising at least one microfabricated capillary 216 (not shown) radiating from the chamber substantially in the direction of the second major axis 207, wherein each of the at least one microfabricated capillary 216 fluidically connects each of the plurality of chambers 214 with an environment outside of each of the plurality of chambers 214, wherein each of the plurality of chambers 214 at least partially overlaps with at least one hole 112 on the central plate first major surface 102A and wherein the microfabricated chip second major surface 212B is adhered to the central plate first major surface 102A;
a first porous membrane 104 having a first porous membrane first surface 104A, and a first porous membrane second surface 104B, the first porous membrane second surface 104B adhered to the microfabricated chip first major surface 212A and sealing at least a portion of the plurality of chambers 214 on the microfabricated chip first major surface 212A, wherein the first porous membrane is impassable for microorganisms;
a perforated film 126 having a perforated film first surface 126A and a perforated film second surface 126B, the perforated film second surface 126B adhered to the first porous membrane first surface 104A, the perforated film 126 having a plurality of holes 127 and at least a portion of the holes 127 in the perforated film 126 at least partially overlap with at least a portion of the holes 102 on the microfabricated chip first major surface 212A;
wherein the first porous membrane 104 together with the perforated film 126 adhered to it can be separated from the microfabricated chip 212;
a removable film 116 having a removable film first surface 116A and a removable film second surface 116B, the removable film first surface 116A adhered to the central plate second major surface 102B.

Making reference to FIG. 18B, the disclosure relates to a device 200 comprising a central plate 102 having a central plate first major surface 102A and a central plate second major surface 102B, the central plate 102 comprising a plurality of holes 112 extending from the central plate first major surface 102A to the central plate second major surface 102B;
a microfabricated chip 212 having a microfabricated chip first major surface 212A, a microfabricated chip second major surface 212B, a first major axis 205 and a second major axis 207 perpendicular to the first major axis 205, a plurality of chambers 214 extending from the microfabricated chip second major surface 212B through to the microfabricated chip first major surface 212A, each of the plurality of chambers 214 comprising at least one microfabricated capillary 216 (not shown) radiating from the chamber substantially in the direction of the second major axis 207, wherein each of the at least one microfabricated capillary 216 fluidically connects each of the plurality of chambers 214 with an environment outside of each of the plurality of chambers 214, wherein each of the plurality of chambers 214 at least partially overlaps with at least one hole 112 on the central plate first major surface 102A and wherein the microfabricated chip second major surface 212B is adhered to the central plate first major surface 102A;
a removable film 116 having a removable film first surface 116A and a removable film second surface 116B, the removable film second surface 116B adhered to the microfabricated chip first major surface 212A;
a second porous membrane 106 having a second porous membrane first surface 106A, and a second porous membrane second surface 106B, the second porous membrane first surface 106A adhered to the central plate second major surface 102B and sealing at least a portion of the plurality of holes 112 on the central plate second major surface 102B, wherein the second porous membrane is impassable for microorganisms; and
a perforated film 128 having a perforated film first surface 128A and a perforated film second surface 128B, the perforated film first surface 128A adhered to the second porous membrane second surface 106B, the perforated film 128 having a plurality of holes 129 and at least a portion of the holes 129 in the perforated film 126 at least partially overlap with at least a portion of the holes 102 on the central plate second major surface 102B;
wherein the second porous membrane 106 together with the perforated film 128 adhered to it can be separated from the central plate 102.

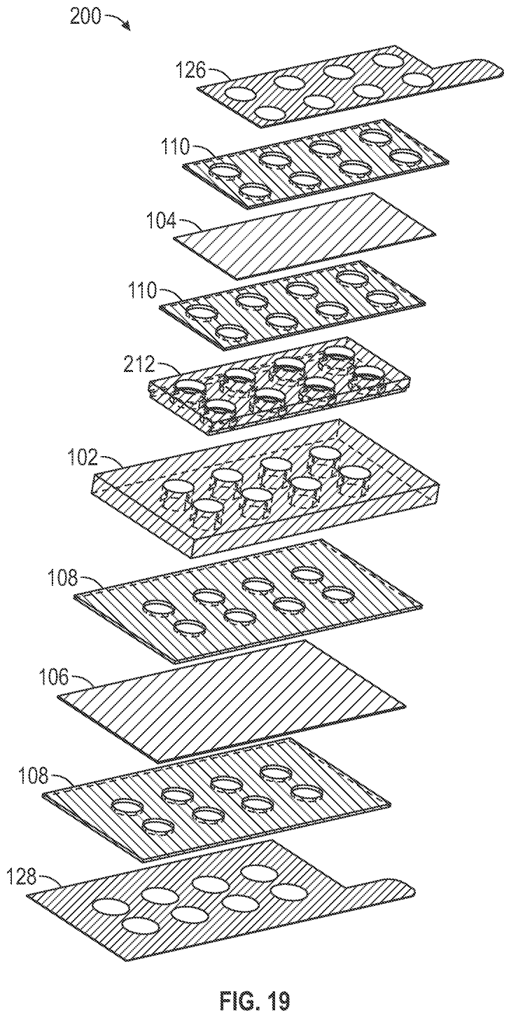

Making reference to FIG. 19, the disclosure relates to a device 200 comprising a central plate 102 having a central plate first major surface 102A and a central plate second major surface 102B, the central plate 102 comprising a plurality of holes 112 extending from the central plate first major surface 102A to the central plate second major surface 102B;
a microfabricated chip 212 having a microfabricated chip first major surface 212A, a microfabricated chip second major surface 212B, a first major axis 205 and a second major axis 207 perpendicular to the first major axis 205, a plurality of chambers 214 extending from the microfabricated chip second major surface 212B through to the microfabricated chip first major surface 212A, each of the plurality of chambers 214 comprising at least one microfabricated capillary 216 (not shown) radiating from the chamber substantially in the direction of the second major axis 207, wherein each of the at least one microfabricated capillary 216 fluidically connects each of the plurality of chambers 214 with an environment outside of each of the plurality of chambers 214, wherein each of the plurality of chambers 214 at least partially overlaps with at least one hole 112 on the central plate first major surface 102A and wherein the microfabricated chip second major surface 212B is adhered to the central plate first major surface 102A;
a first porous membrane 104 having a first porous membrane first surface 104A, and a first porous membrane second surface 104B, the first porous membrane second surface 104B adhered to the microfabricated chip first major surface 212A and sealing at least a portion of the plurality of chambers 214 on the microfabricated chip first major surface 212A, wherein the first porous membrane is impassable for microorganisms;
a perforated film 126 having a perforated film first surface 126A and a perforated film second surface 126B, the perforated film second surface 126B adhered to the first porous membrane first surface 104A, the perforated film 126 having a plurality of holes 127 and at least a portion of the holes 127 in the perforated film 126 at least partially overlap with at least a portion of the chambers 214 on the microfabricated chip first major surface 212A;
wherein the first porous membrane 104 together with the perforated film 126 adhered to it can be separated from the microfabricated chip 212;
a second porous membrane 106 having a second porous membrane first surface 106A, and a second porous membrane second surface 106B, the second porous membrane first surface 106A adhered to the central plate second major surface 102B and sealing at least a portion of the plurality of holes 112 on the central plate second major surface 102B, wherein the second porous membrane is impassable for microorganisms; and
a second perforated film 128 having a second perforated film first surface 128A and a second perforated film second surface 128B, the second perforated film first surface 128A adhered to the second porous membrane second surface 106B, the second perforated film 128 having a plurality of holes 129 and at least a portion of the holes 129 in the perforated film 128 at least partially overlap with at least a portion of the holes 102 on the central plate second major surface 102B;
wherein the second porous membrane 106 together with the perforated film 128 adhered to it can be separated from the central plate 102.

Based on the guidance provided in the instant disclosure, those of ordinary skill in the art can devise additional combinations of central plates, microfabricated chips (when present), porous membrane(s), perforated film(s) (when present), removable film(s) (when present), and thin film(s) that is/are pierceable (when present), even if such combinations may not be explicitly described herein.

The central plates, microfabricated chips (when present), perforated film(s) (when present), porous membrane(s), removable film(s) (when present), solid sheet (when present), solid substrate (when present), and thin film(s) that is/are pierceable (when present), can each be made of any suitable materials. For example, the central plate, solid sheet (when present), and/or solid substrate (when present), can be made from acrylic, polycarbonate, polystyrene, polyethylene, polypropylene, polyethylene terephthalate (PET), acetal plastic, acrylonitrile butadiene styrene (ABS), and the like, and can have any suitable thickness (for example, about 0.5 mm to about 5 mm) and dimensions (for example, about 1 cm to about 20 cm in length and about 1 cm to about 20 cm in width). Alternatively, or in addition, the microfabricated chips can be hot embossed or injection molded in a thermoplastic or can be cast in a silicone elastomer (PDMS) using a photolithography fabricated master mold. Examples of porous membranes that can be used herein include polycarbonate and polyester (PETE) membrane filters with track etched pores by Sterlitech. The pore diameter can be 0.1 μm to make the pores impassable for microorganisms. Examples of materials that can be employed to make the thin film(s) that is/are pierceable (when present) include MYLAR®, SPECTROLENE® film or PROLENE® film, ranging in thickness of from about 3 μm to about 6 μm. Examples of materials that can be employed to make the perforated film(s) (when present), removable film(s) (when present) are polyester, MYLAR®, acrylic, and polycarbonate ranging in thickness from 12.5 μm to 0.5 mm.

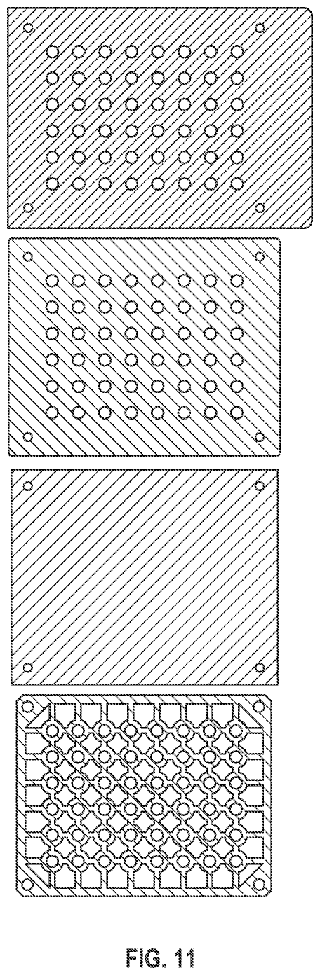

The adhesives used to adhere/bond the various components of the devices described herein (for example, for adhering a porous membrane to a central plate) can be any suitable adhesive, such as a pressure sensitive adhesive transfer tape, for example, 467MP or 468MP by 3M. The adhesive can be used as a frame, as shown in FIGS. 3, 4, 5A, 5B, 6-8, 13-15, 16A, 16B, and 17: or as a perforated adhesive sheets, as shown in FIGS. 1-4, 5A, 5B, 6-11, 13-15, 16A, 16B, 17, 18A, 18B, and 19, wherein the perforations/holes on the adhesive substantially overlap, for example, with the holes on a central plate, chambers on a microfabricated chip, and/or perforated film(s) (when present). The adhesive can be applied to the surface of a central plate after the plate with an array of holes (or wells) is manufactured (using for example, injection molding or laser cutting) or applied to the surface of a plate before holes (or wells) are drilled in the plate. Those of ordinary skill in the art can envision additional methods for adhering/bonding the various components of the devices described herein.

The perforations/holes, when present on certain components of the devices described herein, can have any suitable shape (for example, circular, oval, square, and triangular), size (for example, about 0.5 mm to about 5 mm), and number (for example, an 8×6 array of perforations/holes). See, for example, FIG. 15.

The devices described herein can be assembled by any suitable method. For example, the device described in FIG. 2 can be assembled by applying a perforated layer of adhesive (for example, adhesive transfer tape, such as 3M 467MP) to both sides of the central plate with the array of holes, with the holes in the layer of the adhesive matching the holes on the central plate. A porous membrane with pores impassable for microorganisms but permeable to molecular diffusion can be bonded to one side of the central plate, and a thin pierceable film, which can be non-porous, is bonded to the other side of the central plate.

Before the holes in the central plate are completely sealed, by two porous membranes adhered to both of the central plate major surfaces or by a porous membrane adhered to one major surface and a thin film adhered to the other major surface, the holes in the central plate can be filled with a suspension of microorganisms. The filling of the holes in the central plate with a suspension of microorganisms constitutes inoculation of the device. For example, it can be microorganisms extracted from an environment where the device would be placed later on for an extended incubation. The concentration of the microorganisms can be less than one per hole, such that after the holes are completely sealed, many of the holes contain no more than one microorganism. When the central plate of the device has wells rather than holes (e.g., device in FIG. 5A), the inoculation can be performed before the wells are sealed with a porous membrane. After the device is inoculated, and the holes or wells are completely sealed (with porous membranes, thin films, etc.), these holes or wells become microbial growth chambers.

After the incubation in an environment (for example, soil, mud, or oceanic sediment), the outer surfaces of the device, which can be surfaces of porous membranes, thin pierceable films, and removable (tear-off) film will most likely be covered with microorganisms present in the environment. Cleaning microorganisms from the porous membrane is problematic, because there is always a risk that the cleaning agents would penetrate into the holes of the central plate, killing or harming the microorganisms inside the holes of the central plate, or that the membrane itself would disintegrate. Nevertheless, because the pierceable film is impermeable to liquids, its surface can be cleaned with an organic solvent, bleach, or another sterilizing agent without compromising the microbial cultures inside the holes of the central plate. After the cleaning, the pierceable film can be pierced with, for example, a steel capillary (e.g., a hypodermic needle or a blunt dispensing needle) or a plastic pipette tip and the liquid content of the growth chambers, which are holes (or wells) of the central plate, can be withdrawn without contamination (by microorganisms on the surface of the film).

The central plate itself, comprising a plurality of holes, can be made by injecting molding or by machining acrylic or acetal plastic (e.g., DELRIN R) using laser-cutting. The layer of adhesive, which can be a pressure sensitive adhesive transfer tape, for example, 468MP or 467MP by 3M, can be applied to both sides of the plastic plate before the laser-cutting. Alternatively, the sheets of adhesive transfer tape with, for example, matching arrays of holes (which can be separately laser-cut or produced with some other method) can be applied to the central plate with the array of holes, after the central plate is laser-cut (or manufactured with some other technique).

In one example, the device described in FIG. 2 can be made by laser-cutting a central plate in 0.08″ thick acrylic (about 2 mm, nominal thickness), with an array of holes with the same diameter (for example, between about 0.8 mm to about 5.0 mm) with distances between the adjacent holes being 1.3 mm greater than the diameters of the holes. A layer of 468MP or 467MP adhesive by 3M can be applied with a matching array of holes (with about 0.2 mm greater diameters) to one side of the central plate and used to bond to that side a thin pierceable film that can be, for example, a 3 μm MYLAR® film, a 6 μm SPECTROLENE® film, or a 4 μm PROLENE® film. In the proof-of-concept tests, each these three films, when bonded to the 0.08″ thick acrylic plate with 1.1 mm to 2.5 mm diameter holes using the 467MP or 468MP adhesive tape by 3M, was pierced with a standard 20 μL pipette tip attached to a 1 ml syringe. The piercing did not visibly compromising the adhesion of the film to the central plate in the areas between holes, as judged by the visual inspection under a dissection microscope. Among the three films, the 4 μm PROLENE® film was easiest to pierce and the 0.0023″ thick 467MP adhesive produced better results than the 0.0052″ thick 468MP adhesive.

In another example, the device of FIG. 3 can be assembled to have a porous membrane on one side of the central plate with an array of holes and a pierceable film made of plastic or other material on the other side of the central plate. However, in this modification, the outer side of the thin pierceable film is covered by a tear-off (removable) film, which can be made of plastic or other flexible material. The tear-off film is attached to the pierceable film along a closed strip at the perimeter of one or both of the films, such that the space between the pierceable film and tear-off film is sealed and sterile. A practical way for attaching the tear-off film to the pierceable film is with a layer of adhesive forming a closed frame with inner dimensions greater than the outer dimensions of the array of holes in the central plate. This adhesive frame can be made of an adhesive transfer tape, e.g., 467MP or 468MP by 3M.

The attachment can be made while both the outer surface of the pierceable film and the inner surface of the tear-off film (the side facing the pierceable film) are sterile, that is, before the device is completely assembled and before the device is placed into an environment where microorganisms are present (for example, soil, mud, debris, oceanic sediment). As a result, a sterile cavity/closed volume is formed between the outer surface of the pierceable film and inner surface of the tear-off film, and the outer surface of the pierceable film remains sterile and clean during the incubation, for example, in a soil environment. After the incubation, the tear-off film is removed by pulling, exposing the sterile and clean surface of the pierceable film. Alternatively, the tear-off may be removed from the area facing the array of holes in the central plate by cutting the tear-off film along a line that is enclosing the array of holes and is enclosed by the strip along which the two films are attached to each other (for example, the adhesive frame) and removing the cut-out piece. The pierceable film is then pierced with hollow tubes/capillaries (sharp hypodermic steel needle, blunt steel dispensing needle, plastic pipette tip, etc.) to extract the liquid content of the growth chambers, which are holes or wells in the central plate, one at a time without any cross-contamination.

The three-layer “sandwich” composed of the pierceable film, adhesive frame, and tear-off film can be pre-assembled and attached to one of the major surfaces of the central plate with the array of holes. Alternatively, the pierceable film can first be attached to the central plate on its own and adhesive frame and the tear-off film can be attached to the outer surface of the pierceable film at any point, as long as would-be inner surface of the tear-off film and the outer surface of the pierceable film has been maintained sterile during these steps. In this arrangement, the pierceable film may have pores impassable for microorganisms.

Thus, for example, a device described in FIG. 3 can be made by laser-cutting a central plate in 0.08″ thick acrylic (about 2 mm, nominal thickness), with an array of holes with a diameter from about 0.8 mm to about 5 mm with distances between the adjacent holes being 1.3 mm greater than the diameters of the holes. A layer of 468MP or 467MP pressure sensitive adhesive by 3M with a matching array of holes (with about 0.2 mm greater diameters) can be applied to both sides of the central plate. The application of the adhesive with a precise registration of the holes can be facilitated by making a set of matching auxiliary holes in both the central plate and the adhesive layers and using alignment pins. The adhesive is used to bond a porous membrane (e.g., a polyester or polycarbonate membrane with track etched pores, 0.1 μm in diameter) to one of the major surfaces of the central plate and bonded to bond a pierceable film, e.g., a 6 μm SPECTROLENE® film to the other major surface of the central plate. The same auxiliary holes and alignment pins can then be used to apply to apply to the outer surface of the pierceable film a closed frame made of a pressure-sensitive adhesive, e.g., 0.0023″ thick 467MP by 3M, a with inner dimensions greater than the outer dimensions of the array of holes in the central plate. An adhesive frame can then be used to bond to the outer surface of the pierceable film a tear-off film made out of 0.0005″ to 0.005″ thick PET (MYLAR®) to form a sealed cavity between the two films. In a proof-of-principle test, a 0.0005 thick tear-off film attached with a 467MP adhesive frame was removed from the pierceable 6 μm SPECTROLENE® film by pulling, while the SPECTROLENE® film remained securely attached to the central plate, with all holes in the array reliably sealed. This test was repeated at least 3 times.

So-called self-inoculated devices are also contemplated herein. Such devices comprise a microfabricated chip with an array of chambers, each of which is connected to a proximal end of a single microfabricated capillary channel with a diameter of 0.2-2 μm (see, for example, FIGS. 12A and 12B). The distal end of the capillary is connected to a microfabricated side channel, which is open to one or more of the side surfaces of the chip. All three elements, the chambers, capillaries, and channels, can be engraved on the same surface of the chip. The chip can be cast in a silicone elastomer (PDMS) or hot-embossed in a thermoplastic elastomer using a master mold that may be produced using photolithographic microfabrication. Side channels can appear as simple steps at the bottom of the chip. The chambers and side channels can be deeper than the capillaries and are passable for microorganisms. The side channels can connect the distal end of the capillaries to the outside environment. One purpose of the side channels is to define the length of the capillaries with the high resolution provided by the photolithographic microfabrication (for example, on the order of about 1 μm) as the distance between the chambers and the side channels. An alternative would be a chip without side channels, with the distal ends of the capillaries open to one of the sides of the chip.

The inventive devices have chambers of two types, open at the bottom only and open at both the bottom and the top (FIG. 12A), resulting in an array of openings in the chip. In the latter case, one option would be to cast the chip with a master mold that has features defining the chambers that are as tall (or taller) than the height (thickness) of the chip. Another option would be to use a master mold fabricated with photolithography (or a replica of such a mold) to cast, mold, or hot-emboss a chip with the chambers closed at the top and then post-process the chip by punching or drilling holes through the roofs of the chambers.

Each chip is sealed from the bottom with a central plate that has a polished top surface and an array of holes matching the array of chambers in the chip (FIG. 12A). The size of the holes in the plate and their positions with respect to the chambers in the chip are usually such that each of the microfabricated chambers is connected to a single hole in the central plate (with the hole and the chamber forming a contiguous space) and each of the capillaries is completely sealed from the bottom by the polished surface of the central plate. This way, the lengths of the capillaries are defined with a high precision (for example, on the order of about 1 μm) by the process of microfabrication rather than by the size of the holes and by the alignment of the chip with respect to the central plate, which are normally substantially less precise. A simple exemplary embodiment is with the holes in the central plate having somewhat smaller diameters than the diameters of the openings of the chambers at the bottom surface of the microfabricated chip, and with the precision of the alignment of the holes with respect to the chambers such that the holes are always entirely within the footprint of the openings of the chambers. This way, the edges of the holes in the central plate that may potentially be rough are facing the empty spaces of the chambers. The central plate may be laser cut out of a sheet of acrylic (or acetal plastic) with a polished top surface or injection molded, and the microfabricated chip may be bonded to the polished surface of the central plate using an appropriate chemical treatment (activation) of the surfaces of the chip and central plate. Alternatively, the plate with the array of holes may be cast in a silicone elastomer (PDMS).

The bonding of the microfabricated chip to the central plate can be accomplished without any liquid adhesive (or other liquid or highly deformable material) so as to not reduce (or otherwise change) the diameter of the capillaries of the surface of the chip. The diameter of the capillaries can be defined and maintained with a precision on the order of about 0.1 μm to ensure entrance of microorganisms from the environment into the chambers. For the bonding without adhesives, it can be important that the bottom surface of the chip be brought into close contact with the top surface of the central plate over the entire area of the central plate. To this end, it may be preferable to make the chip, the plate, or both out of a relatively soft material, such as a silicone (PDMS) or thermoplastic elastomer. An exemplary embodiment is to make both the chip and the central plate out of PDMS and bond them together using the oxygen plasma treatment of their surfaces. Alternatively, the central plate can be made of a plastic with a layer of PDMS on its top surface (that can be produced, for example, by spin-coating). If the plate with the array of holes is made out of a soft material, e.g., PDMS, the microfabricated chip may be made out of a hard material.

The bottom side of the central plate has a layer of adhesive (for example, a pressure sensitive adhesive transfer tape from 3M, such as 468MP or 467MP) for sealing the holes in the central plate with a porous membrane, a pierceable film, or a solid sheet. In a basic configuration, with the chambers in the microfabricated chip closed at the top (FIG. 12A), the bottom of the central plate is sealed with a porous membrane which is impassable for microorganisms but permeable to molecular diffusion (for example, FIG. 13). Each of the microfabricated chambers together with the matching hole in the central plate becomes a contiguous growth chamber, which is connected to the outside environment through the porous membrane and the microfabricated capillary. The capillary provides a single entryway into the chamber for microorganisms from the outside environment, whereas the relatively large open area of the porous membrane (for example, about 1% of the total area for track etched membranes) serves the purpose of the molecular exchange between the growth chambers and the outside environment by the molecular diffusion. The growth chambers are filled with a sterile medium before the porous membrane is attached. In the devices with the microfabricated chips open both at the top and bottom (FIG. 12B), a porous membrane may be bonded to the top surface of the chip. In that case, the bottom of the central plate may also be sealed by a pierceable film or a solid sheet.

The devices described herein can be used in a method of growing microorganism cultures. Accordingly, such devices further comprise at least one microorganism inoculum in the wells or holes of the central plates described herein. The at least one microorganism inoculum can be present in at least one of the plurality of central plate wells or holes in the devices described herein. The methods contemplated herein generally relate to growing a microorganism culture, the method comprising: (a) introducing at least one microorganism from an environment into at least one of the plurality of central plate wells or holes of the device of any preceding claim; and (b) incubating the device in the environment. The at least one microorganism can be introduced into the devices described herein by the user or, in the case of the “self-inoculating” devices, the organism can migrate into such devices. The environment comprises, among other things, at least one of soil, mud, oceanic sediment. The incubation time is chosen to enable the at least one microorganism to proliferate and to form a plurality of individual microbial populations each of the plurality of individual microbial populations comprising a large number of microorganisms. After a suitable time, the device is retrieved from the environment. Additional steps in the method include, extracting each of the plurality of individual microbial populations from individual central plate wells or holes without cross-contamination by other microorganisms: sequencing of nucleic acids from each of the plurality of individual microbial populations extracted from individual wells or holes: culturing each of the plurality of microorganism populations in a synthetic or natural medium in a plurality of individual sample containers; and/or analyzing each of the plurality of individual microbial populations and sample containers for the presence of at least one of unique secondary metabolites and small organic molecules (for example, wherein the at least one of unique secondary metabolites and small organic molecules is an antibiotic antibiotics).

In one example, making reference to FIG. 3, the central plate second major surface 102B can be bonded to the thin film first surface 114A, which is a part of a three layer-assembly comprising the thin film 114, the adhesive frame 109, and the removable (tear-off) film 116, with a sealed sterile volume between the thin film second surface 114B and the removable film first surface 116A. As a result, at least a portion of the plurality of the holes 112 on the central plate second major surface 102B are sealed. At least a portion of the plurality of the holes 112 on the central plate second major surface 102B can be filled with a microbial suspension in a suitable medium, such that a majority of the holes contain no microorganisms, only a single microorganism, or only a single species or operational taxonomic unit (OTU) of microorganisms. The central plate first major surface 102A can then be sealed by adhering the first porous membrane second surface 104B. The entire device described in FIG. 3 can then be incubated for a suitable time in the same environment from which the microorganisms in the microbial suspension were extracted. The duration of the incubation time is chosen to enable the microorganisms in the holes of the central plate to proliferate and to form populations of the desired size. After the incubation, the device is retrieved from the environment. The tear-off film 116 can then be removed from the thin film, exposing the sterile second surface of the pierceable film 114B. The thin film 114 can be pierced in an area adjacent to one hole 112 on the central plate with a sterile hollow tube (hypodermic steel needle, pipette tip, etc.) to extract the content of the hole (growth chamber) by aspiration. The piercing is performed without breaking the sealing of other holes on the central plate second surface. The content can be a microbial suspension, and it can be subsequently transferred to a container with a sterile medium for further analysis. The procedure can be repeated for other holes on the central plate second major surface.

In another example, making reference to FIG. 4, the central plate second major surface 102B can be bonded to the first surface 106A of the second porous membrane 106, which is a part of a three layer-assembly comprising the second porous membrane 106, the adhesive frame 109, and the third porous membrane 118, with a sealed sterile volume between the second porous membrane second surface 106B and the third porous membrane first surface 118A. As a result, at least a portion of the plurality of the holes 112 on the central plate second major surface 102B are sealed. At least a portion of the plurality of the holes 112 can be filled with a suspension of microorganisms in a suitable medium, such that a majority of the holes contain no microorganisms, only a single microorganism, or only a single species or operational taxonomic unit (OTU) of microorganisms. The central plate first major surface 102A can then be sealed by adhering the first porous membrane second surface 104B. The entire device described in FIG. 4 can then be incubated for a suitable time in the same environment from which the microorganisms in the microbial suspension were extracted. The duration of the incubation time is chosen to enable the microorganisms in the holes of the central plate to proliferate and to form populations of the desired size. After the incubation, the device is retrieved from the environment. The third porous membrane 118 can be cut along the cutting line 119, exposing the sterile second porous membrane second surface 106B. The second porous membrane 106 can be pierced in an area adjacent to one hole 112 on the central plate with a sterile hollow tube (hypodermic steel needle, pipette tip, etc.) to extract the content of the hole (growth chamber) by aspiration. The piercing is performed without breaking the sealing of other holes on the central plate second surface 102B. The content can be a microbial suspension, and it can be subsequently transferred to a container with a sterile medium for further analysis. The procedure can be repeated for other holes on the central plate second major surface.

“Further analysis,” as used herein, includes culturing microorganisms from each of the plurality of individual samples/containers in synthetic and natural medium. “Further analysis,” as used herein, also includes the sequencing of nucleic acids of the microorganisms from the individual samples/containers. “Further analysis,” also includes analyzing each of the plurality of individual samples for unique secondary metabolites, small organic molecules, including molecules that can be practically useful, e.g., as antibiotics. “Further analysis” also includes identifying and isolating only microorganisms that produce those unique secondary metabolites, small organic molecules, including molecules that can be practically useful, e.g., as antibiotics.

In another example, this time making reference to FIG. 14, the central plate 102, the microfabricated chip 212, the second porous membrane 106, and the third porous membrane 118 are adhered to one another as shown and as described herein. There is least one sealed and sterile volume between the second porous membrane second surface 106B and third porous membrane first surface 118A, wherein at least a portion of the at least one sealed and sterile volume is adjacent to the holes 112 on the central plate 102. Each of the plurality of chambers 214 (and capillaries 216) and the holes 112 on the central plate are filled with a liquid medium, a solid medium or a liquid medium that solidifies. The first porous membrane second surface 104B is then adhered to the microfabricated chip first major surface 212A, sealing at least a portion of the plurality of chambers 214. In this example, the plurality of chambers 214 and the holes 112 on the central plate are first filled with a sterile medium and do not comprise any microorganisms. The entire device described in FIG. 14 can then be incubated for a suitable time in an environment that is expected to contain microorganisms, for example, a soil, mud, oceanic sediment, etc. The duration of the incubation time is empirically selected to enable the microorganisms from the environment to penetrate into the chambers of the device through the capillaries, to proliferate in the chambers, forming populations of the desired size. The third porous membrane 118 can then be cut along the cutting line 119 and cut-out part of the third porous membrane 118 can be removed to expose the second porous membrane second surface 106B, which is sterile. The second porous membrane 106 can be pierced in an area adjacent to one hole 112 on the central plate with a sterile hollow tube (hypodermic steel needle, pipette tip, etc.) to extract the content of the growth chamber (hole) by aspiration. The piercing is performed without breaking the sealing of other holes on the central plate second surface 102B. The content can be a microbial suspension, and it can be subsequently transferred to a container with a sterile medium for further analysis. The procedure can be repeated for other holes on the central plate second major surface.

Also contemplated herein are kits comprising the components (for example, central plate(s), microfabricated chip(s), perforated film(s), porous membrane(s), removable film(s), solid sheet(s), solid substrate(s), thin film(s), and adhesives used to adhere/bond the various components of the devices) for assembling the various devices described herein either in a single container (for example, a bag) or in separate containers: the devices described herein at least partially assembled, accompanied by the components necessary to complete their assembly, each in the same container or separate containers: or the devices described herein fully assembled. The kits can further comprise instructions for at least one of the use of the devices or for the assembly of the devices. The kits can further comprise one or more reagents including, for example, liquid medium, a liquid medium that solidifies, or ingredients for making this media.

The specific methods, devices and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications can be made to the invention disclosed herein without departing from the scope and spirit of the invention.

The invention illustratively described herein suitably can be practiced in the absence of any element or elements, or limitation or limitations, which is not specifically disclosed herein as essential. The methods and processes illustratively described herein suitably can be practiced in differing orders of steps, and the methods and processes are not necessarily restricted to the orders of steps indicated herein or in the claims.

Under no circumstances can the patent be interpreted to be limited to the specific examples or embodiments or methods specifically disclosed herein. Under no circumstances can the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed can be resorted to by those skilled in the art, and that such modifications and variations are considered to be within the scope of this invention as defined by the appended claims and statements of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein. In addition, where features or aspects of the invention are described in terms of Markush groups, those skilled in the art will recognize that the invention is also thereby described in terms of any individual member or subgroup of members of the Markush group.

The disclosure provides for the following example Statements, the numbering of which is not to be construed as designating levels of importance:

1. A device comprising:
a central plate 102 having a central plate first major surface 102A and a central plate second major surface 102B, the central plate 102 comprising a plurality of holes 112 extending from the central plate first major surface 102A to the central plate second major surface 102B; and
a first porous membrane 104 having a first porous membrane first surface 104A, and a first porous membrane second surface 104B, the first porous membrane second surface 104B adhered to the central plate first major surface 102A and sealing at least a portion of the plurality of holes 112 on the central plate first major surface 102A, wherein the first porous membrane is impassable for microorganisms; and
a thin film 114, which may be pierceable, having a thin film first surface 114A and a thin film second surface 114B, the thin film first surface 114A adhered to the central plate second major surface 102B and sealing at least a portion of the plurality of holes 112 on the central plate second major surface 102B;
wherein:
the thin film second surface 114B can be cleaned and sterilized, and the thin film 114 can be pierced in an area adjacent to one hole 112 or a plurality of holes 112 on the central plate without breaking the sealing of other holes on the central plate second major surface.

2. A device comprising:
a central plate 102 having a central plate first major surface 102A and a central plate second major surface 102B, the central plate 102 comprising a plurality of wells 113 on the central plate first major surface 102A;
a first porous membrane 104 having a first porous membrane first surface 104A, and a first porous membrane second surface 104B, the first porous membrane second surface 104B adhered to the central plate first major surface 102A and sealing at least a portion of the plurality of wells 113 on the central plate first major surface 102A, wherein the first porous membrane 104 is impassable for microorganisms;
a fourth porous membrane 120 having a fourth porous membrane first surface 120A, and a fourth porous membrane second surface 120B, the fourth porous membrane second surface 120B adhered to the first porous membrane first surface 104A and covering an area with at least a portion of the plurality of wells 113 on the central plate first major surface 102A that are sealed by the first porous membrane 104, wherein the fourth porous membrane 120 is impassable for microorganisms; and
at least one sealed and sterile volume between the first porous membrane first surface 104A and second porous membrane second surface 120B, wherein at least a portion of the at least one sealed and sterile volume is adjacent to at least one well 113 on the central plate 102 that is sealed by the first porous membrane 104.

3. A device comprising:
a central plate 102 having a central plate first major surface 102A and a central plate second major surface 102B, the central plate 102 comprising a plurality of holes 112 extending from the central plate first major surface 102A to the central plate second major surface 102B;
a first porous membrane 104 having a first porous membrane first surface 104A, and a first porous membrane second surface 104B, the first porous membrane second surface 104B adhered to the central plate first major surface 102A and sealing at least a portion of the plurality of holes 112 on the central plate first major surface 102A, wherein the first porous membrane 104 is impassable for microorganisms;
a fourth porous membrane 120 having a fourth porous membrane first surface 120A, and a fourth porous membrane second surface 120B, the fourth porous membrane second surface 120B adhered to the first porous membrane first surface 104A and covering an area with at least a portion of the plurality of holes on the central plate first major surface 102A that are sealed by the first porous membrane 104, wherein the fourth porous membrane 120 is impassable for microorganisms; and
at least one sealed and sterile volume between the first porous membrane first surface 104A and second porous membrane second surface 120B, wherein at least a portion of the at least one sealed and sterile volume is adjacent to at least one hole 112 on the central plate first major surface 102A that is sealed by the first porous membrane 104.

4. A device comprising:
a central plate 102 having a central plate first major surface 102A and a central plate second major surface 102B, the central plate 102 comprising a plurality of holes 112 extending from the central plate first major surface 102A to the central plate second major surface 102B;
a first porous membrane 104 having a first porous membrane first surface 104A, and a first porous membrane second surface 104B, the first porous membrane second surface 104B adhered to the central plate first major surface 102A and sealing at least a portion of the plurality of holes 112 on the central plate first major surface 102A, wherein the first porous membrane is impassable for microorganisms;
a thin film 114 having a thin film first surface 114A and a thin film second surface 114B, the thin film first surface 114A adhered to the central plate second major surface 102B and sealing at least a portion of the plurality of holes 112 on the central plate second major surface 102B, wherein the thin film 114 can be pierced in an area adjacent to one hole 112 or a plurality of holes 112 on the central plate 102 without breaking the sealing of other holes 112 on the central plate second major surface 102B;
a removable film 116 having a removable film first surface 116A and a removable film second surface 116B, the removable film first surface 116A adhered to the thin film second surface 114B and covering an area with at least a portion of the plurality of holes 112 on the central plate second major surface 102B that are sealed by the thin film 114; at least one sealed and sterile volume between the thin film second surface 114B and the removable film first surface 116A, and at least a portion of the at least one sealed and sterile volume is adjacent to at least one hole 112 on the central plate second major surface 102B that is sealed by the thin film 114; and
the removable film 116 can be separated from the thin film 114 without unsealing the sealing of the holes on the central plate second major surface 102B that are sealed by the thin film 114.

5. A device comprising:
a central plate 102 having a central plate first major surface 102A and a central plate second major surface 102B, the central plate 102 comprising a plurality of holes 112 extending from the central plate first major surface 102A to the central plate second major surface 102B;
a first porous membrane 104 having a first porous membrane first surface 104A, and a first porous membrane second surface 104B, the first porous membrane second surface 104B adhered to the central plate first major surface 102A and sealing at least a portion of the plurality of holes 112 on the central plate first major surface 102A, wherein the first porous membrane is impassable for microorganisms; and
a perforated film 126 having a perforated film first surface 126A and a perforated film second surface 126B, the perforated film second surface 126B adhered to the first porous membrane first surface 104A, the perforated film 126 having a plurality of holes 127 and at least a portion of the holes 127 in the perforated film 126 at least partially overlap with at least a portion of the holes 102 on the central plate first major surface 102A;
wherein the first porous membrane 104 together with the perforated film 126 adhered to it can be separated from the central plate 102.

6. The device of Statement 5, further comprising a removable film 116 having a removable film first surface 116A and a removable film second surface 116B, the removable film first surface 116A adhered to the central plate second major surface 102B.

7. The device of Statement 5, further comprising a second porous membrane 106 having a second porous membrane first surface 106A, and a second porous membrane second surface 106B, the second porous membrane first surface 106A adhered to the central plate second major surface 102B and sealing at least a portion of the plurality of holes 112 on the central plate second major surface 102B, wherein the second porous membrane is impassable for microorganisms; and
a second perforated film 128 having a second perforated film first surface 128A and a second perforated film second surface 128B, the second perforated film first surface 126A adhered to the second porous membrane second surface 106B, the second perforated film 128 having a plurality of holes 129 and at least a portion of the holes 129 in the second perforated film 128 at least partially overlap with at least a portion of the holes 102 on the central plate second major surface 102B.

8. The device of any preceding Statement, further comprising a microorganism inoculum in the wells or holes of the central plate.

9. The device of Statement 8, wherein the microorganism inoculum is present in at least one of the plurality of central plate wells or holes.

10. The device of Statement 8, wherein the content of the central plate wells or holes comprises a liquid medium, a solid medium or a liquid medium that solidifies.

11. A method of growing a microorganism culture, the method comprising:
(a) introducing at least one microorganism from an environment into at least one of the plurality of central plate wells or holes of the device of any preceding Statement; and
(b) incubating the device in the environment.

12. The method of Statement 11, wherein the environment comprises at least one of soil, mud, oceanic sediment.

13. The method of Statement 11, wherein an incubation time is chosen to enable the at least one microorganism to proliferate and to form a plurality of individual microbial populations each of the plurality of individual microbial populations comprising a large number of microorganisms; or an incubation time is chosen to enable the at least one microorganism to proliferate and to form a population comprising multiple microorganisms.

14. The method of Statement 11 or 13, further comprising retrieving the device from the environment.

15. The method of Statement 14, further comprising extracting each of the plurality of individual microbial populations from individual central plate wells or holes without cross-contamination by other microorganisms; or
extracting the plurality of individual populations of microorganisms from individual central plate wells or holes one by one without cross-contamination by microorganisms from other central plate wells or holes and by other microorganisms.

16. The method of Statement 14, further comprising sequencing of nucleic acids from each of the plurality of individual microbial populations extracted from individual wells or holes; or
sequencing of nucleic acids from each of the plurality of individual populations of microorganisms extracted from individual wells or holes.

17. The method of Statement 14, further comprising culturing each of the plurality of microorganism populations in a synthetic or natural medium in a plurality of individual sample containers; or
culturing each of the plurality of populations of microorganisms in a synthetic or natural medium in a plurality of individual sample containers.

18. The method of Statement 15 or 17, further comprising analyzing each of the plurality of individual microbial populations and sample containers for the presence of at least one of unique secondary metabolites and small organic molecules; or
analyzing each of the plurality of individual populations of microorganisms or the content of the sample containers for the presence of at least one of secondary metabolites and small organic molecules.

19. The method of Statement 18, wherein the at least one of unique secondary metabolites and small organic molecules is an antibiotic; or
wherein the at least one of secondary metabolites and small organic molecules is an antibiotic.

20. A kit comprising the device of Statements 1-10.

1. A device comprising:
a central plate 102 having a central plate first major surface 102A and a central plate second major surface 102B, the central plate 102 comprising a plurality of holes 112 extending from the central plate first major surface 102A to the central plate second major surface 102B; and
a first porous membrane 104 having a first porous membrane first surface 104A, and a first porous membrane second surface 104B, the first porous membrane second surface 104B adhered to the central plate first major surface 102A and sealing at least a portion of the plurality of holes 112 on the central plate first major surface 102A, wherein the first porous membrane is impassable for microorganisms; and
a thin film 114, which may be pierceable, having a thin film first surface 114A and a thin film second surface 114B, the thin film first surface 114A adhered to the central plate second major surface 102B and sealing at least a portion of the plurality of holes 112 on the central plate second major surface 102B;
wherein:
the thin film second surface 114B can be cleaned and sterilized, and the thin film 114 can be pierced in an area adjacent to one hole 112 or a plurality of holes 112 on the central plate without breaking the sealing of other holes on the central plate second major surface.

2. A device comprising:
a central plate 102 having a central plate first major surface 102A and a central plate second major surface 102B, the central plate 102 comprising a plurality of wells 113 on the central plate first major surface 102A;
a first porous membrane 104 having a first porous membrane first surface 104A, and a first porous membrane second surface 104B, the first porous membrane second surface 104B adhered to the central plate first major surface 102A and sealing at least a portion of the plurality of wells 113 on the central plate first major surface 102A, wherein the first porous membrane 104 is impassable for microorganisms;
a fourth porous membrane 120 having a fourth porous membrane first surface 120A, and a fourth porous membrane second surface 120B, the fourth porous membrane second surface 120B adhered to the first porous membrane first surface 104A and covering an area with at least a portion of the plurality of wells 113 on the central plate first major surface 102A that are sealed by the first porous membrane 104, wherein the fourth porous membrane 120 is impassable for microorganisms; and
at least one sealed and sterile volume between the first porous membrane first surface 104A and second porous membrane second surface 120B, wherein at least a portion of the at least one sealed and sterile volume is adjacent to at least one well 113 on the central plate 102 that is sealed by the first porous membrane 104.

3. A device comprising:
a central plate 102 having a central plate first major surface 102A and a central plate second major surface 102B, the central plate 102 comprising a plurality of holes 112 extending from the central plate first major surface 102A to the central plate second major surface 102B;
a first porous membrane 104 having a first porous membrane first surface 104A, and a first porous membrane second surface 104B, the first porous membrane second surface 104B adhered to the central plate first major surface 102A and sealing at least a portion of the plurality of holes 112 on the central plate first major surface 102A, wherein the first porous membrane 104 is impassable for microorganisms;
a fourth porous membrane 120 having a fourth porous membrane first surface 120A, and a fourth porous membrane second surface 120B, the fourth porous membrane second surface 120B adhered to the first porous membrane first surface 104A and covering an area with at least a portion of the plurality of holes on the central plate first major surface 102A that are sealed by the first porous membrane 104, wherein the fourth porous membrane 120 is impassable for microorganisms; and
at least one sealed and sterile volume between the first porous membrane first surface 104A and second porous membrane second surface 120B, wherein at least a portion of the at least one sealed and sterile volume is adjacent to at least one hole 112 on the central plate first major surface 102A that is sealed by the first porous membrane 104.

4. A device comprising:
a central plate 102 having a central plate first major surface 102A and a central plate second major surface 102B, the central plate 102 comprising a plurality of holes 112 extending from the central plate first major surface 102A to the central plate second major surface 102B;
a first porous membrane 104 having a first porous membrane first surface 104A, and a first porous membrane second surface 104B, the first porous membrane second surface 104B adhered to the central plate first major surface 102A and sealing at least a portion of the plurality of holes 112 on the central plate first major surface 102A, wherein the first porous membrane is impassable for microorganisms;
a thin film 114 having a thin film first surface 114A and a thin film second surface 114B, the thin film first surface 114A adhered to the central plate second major surface 102B and sealing at least a portion of the plurality of holes 112 on the central plate second major surface 102B, wherein the thin film 114 can be pierced in an area adjacent to one hole 112 or a plurality of holes 112 on the central plate 102 without breaking the sealing of other holes 112 on the central plate second major surface 102B;
a removable film 116 having a removable film first surface 116A and a removable film second surface 116B, the removable film first surface 116A adhered to the thin film second surface 114B and covering an area with at least a portion of the plurality of holes 112 on the central plate second major surface 102B that are sealed by the thin film 114; at least one sealed and sterile volume between the thin film second surface 114B and the removable film first surface 116A, and at least a portion of the at least one sealed and sterile volume is adjacent to at least one hole 112 on the central plate second major surface 102B that is sealed by the thin film 114; and
the removable film 116 can be separated from the thin film 114 without unsealing the sealing of the holes on the central plate second major surface 102B that are sealed by the thin film 114.

5. A device comprising:
a central plate 102 having a central plate first major surface 102A and a central plate second major surface 102B, the central plate 102 comprising a plurality of holes 112 extending from the central plate first major surface 102A to the central plate second major surface 102B;
a first porous membrane 104 having a first porous membrane first surface 104A, and a first porous membrane second surface 104B, the first porous membrane second surface 104B adhered to the central plate first major surface 102A and sealing at least a portion of the plurality of holes 112 on the central plate first major surface 102A, wherein the first porous membrane is impassable for microorganisms; and
a perforated film 126 having a perforated film first surface 126A and a perforated film second surface 126B, the perforated film second surface 126B adhered to the first porous membrane first surface 104A, the perforated film 126 having a plurality of holes 127 and at least a portion of the holes 127 in the perforated film 126 at least partially overlap with at least a portion of the holes 102 on the central plate first major surface 102A;
wherein the first porous membrane 104 together with the perforated film 126 adhered to it can be separated from the central plate 102.

6. The device of claim 5, further comprising a removable film 116 having a removable film first surface 116A and a removable film second surface 116B, the removable film first surface 116A adhered to the central plate second major surface 102B.

7. The device of claim 5, further comprising a second porous membrane 106 having a second porous membrane first surface 106A, and a second porous membrane second surface 106B, the second porous membrane first surface 106A adhered to the central plate second major surface 102B and sealing at least a portion of the plurality of holes 112 on the central plate second major surface 102B, wherein the second porous membrane is impassable for microorganisms; and
a second perforated film 128 having a second perforated film first surface 128A and a second perforated film second surface 128B, the second perforated film first surface 126A adhered to the second porous membrane second surface 106B, the second perforated film 128 having a plurality of holes 129 and at least a portion of the holes 129 in the second perforated film 128 at least partially overlap with at least a portion of the holes 112 on the central plate second major surface 102B.

8. The device of claim 1, further comprising a microorganism inoculum in the wells or holes of the central plate.

9. The device of claim 8, wherein the microorganism inoculum is present in at least one of the plurality of central plate wells or holes.

10. The device of claim 8, wherein the content of the central plate wells or holes comprises a liquid medium, a solid medium or a liquid medium that solidifies.

11. A method of growing a microorganism culture, the method comprising:
(a) introducing at least one microorganism from an environment into at least one of the plurality of central plate wells or holes of the device of claim 1; and
(b) incubating the device in the environment.

12. The method of claim 11, wherein the environment comprises at least one of soil, mud, oceanic sediment.

13. The method of claim 11, wherein an incubation time is chosen to enable the at least one microorganism to proliferate and to form a plurality of individual microbial populations each of the plurality of individual microbial populations comprising a large number of microorganisms.

14. The method of claim 13, further comprising retrieving the device from the environment.

15. The method of claim 14, further comprising extracting each of the plurality of individual microbial populations from individual central plate wells or holes without cross-contamination by other microorganisms.

16. The method of claim 14, further comprising sequencing of nucleic acids from each of the plurality of individual microbial populations extracted from individual wells or holes.

17. The method of claim 14, further comprising culturing each of the plurality of microorganism populations in a synthetic or natural medium in a plurality of individual sample containers.

18. The method of claim 15, further comprising analyzing each of the plurality of individual microbial populations and sample containers for the presence of at least one of unique secondary metabolites and small organic molecules.

19. The method of claim 18, wherein the at least one of unique secondary metabolites and small organic molecules is an antibiotic.

20. A kit comprising the device as in claim 1.
